(19) United States
(12) Patent Application Publication
(10) Pub. No.: US 2004/0082543 A1
(43) Pub. Date: Apr. 29, 2004

(54) Compositions of cyclooxygenase-2 selective inhibitors and NMDA receptor antagonists for the treatment or prevention of neuropathic pain

(75) Inventor: Raymond Y. Cheung, Bridgewater, NJ (US)

Correspondence Address: Kathryn J. Doty, SENNIGER, POWERS, LEAVITT & ROEDEL, One Metropolitan Square, 16th Floor, St. Louis, MO, 63103, US

(73) Assignee: Pharmacia Corporation

(21) Appl. No.: 10/282,660

(22) Filed: Oct. 29, 2002

(51) Int. Cl. A61K 31/675; A61K 31/498; A61K 31/50; A61K 31/415; A61K 31/18

(52) U.S. Cl. 514/80; 514/247; 514/249; 514/378; 514/406; 514/602; 514/312; 514/183

(57) ABSTRACT

The present invention provides compositions and methods to treat or prevent neuropathic pain in a subject using a combination of a COX-2 selective inhibitor and a NMDA receptor antagonist.

### FIELD OF THE INVENTION

The present invention relates to compositions and methods for the treatment or prevention of neuropathic pain in a subject using a combination of a COX-2 selective inhibitor and a NMDA receptor antagonist.

### BACKGROUND OF THE INVENTION

Pain is a sensory experience distinct from sensations of touch, pressure, heat and cold. It is often described by sufferers by such terms as bright, dull, aching, pricking, cutting or burning and is generally considered to include both the original sensation and the reaction to that sensation. This range of sensations, as well as the variation in perception of pain by different individuals, renders a precise definition of pain difficult, however, many individuals suffer with severe and continuous pain.

Pain can be “caused” by the stimulation of nociceptive receptors and transmitted over intact neural pathways, in which case such pain is termed “nociceptive” pain. Pain that is caused by damage to neural structures is often manifest as a neural supersensitivity or hyperalgesia and is termed “neuropathic” pain. Neuropathic pain may be caused by prolonged peripheral nociceptive input that results in central sensitization with spinal and cortical reorganization.

Approximately $80 billion dollars are spent annually in the US to treat chronic pain, from which 80 million Americans suffer. Currently, most of the drugs used to treat neuropathic pain were developed for other uses and their non-selectivity leads to side-effects that greatly limit their usefulness. Current therapy for chronic pain relies on pharmacological therapies with NSAIDs, opioid analgesics and co-analgesic drugs, such as antidepressants, anticonvulsants, and calcium channel blockers. Invasive techniques are also used, such as peripheral and central nerve blockade using local anaesthetic agents and corticosteroid adjuvants (M. J. Abrahams, et al., Emerging Drugs, 5(4), 385-413 (2000)). However, adverse side effects limit treatment efficacy, for example, the gastrointestinal and renal effects of NSAIDs and the sedative effects of antidepressants.

Of all of the opioid analgesics, morphine remains the most widely used, but, in addition to its therapeutic properties, it has a number of drawbacks including respiratory depression, decreased gastrointestinal motility (resulting in constipation), nausea and vomiting. Tolerance and physical dependence also limit the clinical uses of opioid compounds. Most existing drugs provide only temporary relief from pain and must be taken consistently on a daily or weekly basis. With disease progression, the amount of medication needed to alleviate the pain often increases, thus increasing the potential for adverse side effects.

One emerging class of therapeutic agents for the treatment of neuropathic pain is NMDA receptor antagonists. NMDA receptors are defined by the binding of N-methyl-D-aspartate (NMDA) and comprise a receptor/ion channel complex with several different identified binding domains. The activation of the NMDA receptor following peripheral tissue or nerve injury is thought to play a significant tole in long-term plastic changes in the central nervous system leading to central sensitization and neuropathic pain. However, many NMDA antagonists cause numerous side effects, such as memory impairment, pyschotomimetic effects, ataxia and motor incoordination, since they impair the normal synaptic transmission as well as the pathological activation of the NMDA receptor (C. G. Parsons, European Journal of Pharmacology, 429, 71-78 (2001)).

Prostaglandins play a major role in the inflammation process and the inhibition of prostaglandin production, especially production of PGG₂, PGH₂ and PGE₂ has been a common target of anti-inflammatory drug discovery. However, common non-steroidal anti-inflammatory drugs (NSAIDs) that are active in reducing the prostaglandin-induced pain and swelling associated with the inflammation process are also active in affecting other prostaglandin-regulated processes not associated with the inflammation process. Thus, use of high doses of most common NSAIDs can produce severe side effects, including life threatening ulcers that limit their therapeutic potential. Previous NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase-2 (COX-2)” or “prostaglandin G/H synthase II”) provides a viable target of inhibition that more effectively reduces inflammation and produces fewer and less drastic side effects.

Recent results indicate that the induction of COX-2 in the central nervous system, leading to the production of prostaglandins, followed by central sensitization, is involved in the development of neuropathic pain (W. Ma, et al., European Journal of Neuroscience, 15, 1037-1047 (2002)). Intrathecal injection of a COX-2 inhibitor into rats having peripheral nerve injury significantly reversed tactile allodynia for a period of time (W. Ma, Brain Research, 937, 94-99 (2002)). Thus spinal COX-2 may play an important role in the development and maintenance of neuropathic pain.

WO 00/51685 describes the combination of tramadol and a selective COX-2 inhibitor for the treatment of pain, inflammation, and neurological disorders.

WO 98/50075 describes the combination of NMDA blockers and COX-2 inhibitors for the alleviation of pain.

WO 99/25382 describes the combination of NMDA antagonists and COX-2 inhibitors for the treatment of pain and inflammatory phenomena.

WO 99/44640 describes the combination of a selective NMDA NR2B antagonist and a COX-2 inhibitor for the treatment or prevention of pain or nociception.

WO 00/29023 describes a method for alleviating a pain state utilizing a NMDA blocker and a COX-2 inhibitor.

WO 01/38311 describes pyrimidine derivatives as selective COX-2 inhibitors that may be used in combination with NMDA modulators for the treatment of pain.

WO 01/40216 describes heterocycloalkylsulfonyl pyrazole derivatives as anti-inflammatory and analgesic agents that may be used in combination with NMDA antagonists for the treatment of pain.

EP 1104760 describes sulfamoylheteroaryl pyrazole compounds as anti-inflammatory and analgesic agents that may be used in combination with NMDA antagonists for the treatment of pain.

EP 1104759 describes heteroaryl phenyl pyrazole compounds as anti-inflammatory and analgesic agents that may be used in combination with NMDA antagonists for the treatment of pain.

EP 1104758 describes acetylene derivatives as anti-inflammatory and analgesic agents that may be used in combination with NMDA antagonists for the treatment of pain.

WO 01/64669 describes pyrazole ether derivatives as anti-inflammatory and analgesic agents that may be used in combination with NMDA antagonists for the treatment of pain.

EP 1142889 describes pyrazole derivatives as anti-inflammatory and analgesic agents that may be used in combination with NMDA antagonists for the treatment of pain.

### SUMMARY OF THE INVENTION

Among several aspects of the present invention is provided a composition comprising a COX-2 selective inhibitor and a NMDA antagonist, wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a compound having a diarylmethylidenefuran, a compound having a 2-phenylaminobenzene acetic acid, a compound having a chromene, and parecoxib or a pharmaceutically acceptable salt, prodrug or isomer thereof; and

wherein the NMDA antagonist is selected from the group consisting of

(−)-6,7-dichloro-1,4-dihydro-5-[3-(methoxymethyl)-5-(3-pyridinyl)-4H-1,2,4-triazol-4-yl]-2,3-quinoxalinedione,

(2R,4S)-rel-5,7-dichloro-1,2,3,4-tetrahydro-4-[[(phenylamino)carbonyl]amino]-2-quinolinecarboxylic acid,

(2R,6S)-1,2,3,4,5,6-hexahydro-3-[(2S)-2-methoxypropyl]-6,11,11-trimethyl-2,6-methano-3-benzazocin-9-01,

(3E)-2-amino-4-(phosphonomethyl)-3-heptenoic acid,

(3R,4S)-rel-3,4-dihydro-3-[4-hydroxy-4-(phenylmethyl)-1-piperidinyl]-2H-1-benzopyran-4,7-diol,

(3S,4aR,6S,8aR)-decahydro-6-(phosphonomethyl)-3-isoquinolinecarboxylic acid,

3,6,7-tetrahydro-2,3-dioxo-N-phenyl-1H,5H-pyrido[1,2,3-de]quinoxaline-5-acetamide,

(αR)-α-amino-5-chloro-1-(phosphonomethyl)-1H-benzimidazole-2-propanoic acid,

[2-(8,9-dioxo-2,6-diazabicyclo[5.2.0]non-1(7)-en-2-yl)ethyl]-phosphonic acid,

[5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride,

1,4-dihydro-6-methyl-5-[(methylamino)methyl]-7-nitro-2,3-quinoxalinedione,

1-[2-(4-hydroxyphenoxy)ethyl]-4-[(4-methylphenyl)methyl]-4-piperidinol, hydrochloride,

1-[4-(1H-imidazol-4-yl)-3-butynyl]-4-(phenylmethyl)-piperidine,

1-aminocyclopentane-carboxylic acid,

2-[(2,3-dihydro-1H-inden-2-yl)amino]-acetamide, monohydrochloride,

2-hydroxy-5-[[(pentafluorophenyl)methyl]amino]-benzoic acid,

2-methyl-6-(phenylethynyl)-pyridine,

3-(phosphonomethyl)-L-phenylalanine,

3-[(1E)-2-carboxy-2-phenylethenyl]-4,6-dichloro-1H-indole-2-carboxylic acid,

4,6-dichloro-3-[(E)-(2-oxo-1-phenyl-3-pyrrolidinylidene)methyl]-1H-indole-2-carboxylic acid,

6-chloro-2,3,4,9-tetrahydro-9-methyl-2,3-dioxo-1H-indeno[1,2-b]pyrazine-9-acetic acid,

7-chlorothiokynurenic acid,

8-chloro-2,3-dihydropyridazino[4,5-b]quinoline-1,4-dione 5-oxide salt with 2-hydroxy-N,N,N-trimethyl-ethanaminium,

aptiganel,

besonprodil,

budipine,

conantokin G,

delucemine,

dexanabinol,

felbamate,

fluorofelbamate,

gacyclidine,

glycine,

ipenoxazone,

kaitocephalin,

lanicemine,

licostinel,

midafotel,

milnacipran,

N′-[2-chloro-5-(methylthio)phenyl]-N-methyl-N-[3-(methylthio)phenyl]-guanidine,

N′-[2-chloro-5-(methylthio)phenyl]-N-methyl-N-[3-[(R)-methylsulfinyl]phenyl]-guanidine,

neramexane,

orphenadrine,

remacemide,

topiramate,

α-amino-2-(2-phosphonoethyl)-cyclohexanepropanoic acid, and

α-amino-4-(phosphonomethyl)-benzeneacetic acid

or a pharmaceutically acceptable salt thereof.

Yet another aspect of the invention provides a method for the treatment or prevention of neuropathic pain in a subject, the method comprising administering to the subject a COX-2 selective inhibitor and a NMDA antagonist,

wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a compound having a diarylmethylidenefuran, a compound having a 2-phenylaminobenzene acetic acid, a compound having a chromene, and parecoxib or a pharmaceutically acceptable salt, prodrug or isomer thereof; and

wherein the NMDA antagonist is selected from the group consisting of

(−)-6,7-dichloro-1,4-dihydro-5-[3-(methoxymethyl)-5-(3-pyridinyl)-4H-1,2,4-triazol-4-yl]-2,3-quinoxalinedione,

(2R,4S)-rel-5,7-dichloro-1,2,3,4-tetrahydro-4-[[(phenylamino)carbonyl]amino]-2-quinolinecarboxylic acid,

(2R,6S)-1,2,3,4,5,6-hexahydro-3-[(2S)-2-methoxypropyl]-6,11,11-trimethyl-2,6-methano-3-benzazocin-9-ol,

(3E)-2-amino-4-(phosphonomethyl)-3-heptenoic acid,

(3R,4S)-rel-3,4-dihydro-3-[4-hydroxy-4-(phenylmethyl)-1-piperidinyl]-2H-1-benzopyran-4,7-diol,

(3S,4aR,6S,8aR)-decahydro-6-(phosphonomethyl)-3-isoquinolinecarboxylic acid,

3,6,7-tetrahydro-2,3-dioxo-N-phenyl-1H,5H-pyrido[1,2,3-de]quinoxaline-5-acetamide,

(αR)-α-amino-5-chloro-1-(phosphonomethyl)-1H-benzimidazole-2-propanoic acid,

[2-(8,9-dioxo-2,6-diazabicyclo[5.2.0]non-1(7)-en-2-yl)ethyl]-phosphonic acid,

[5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride,

1,4-dihydro-6-methyl-5-[(methylamino)methyl]-7-nitro-2,3-quinoxalinedione,

1-[2-(4-hydroxyphenoxy)ethyl]-4-[(4-methylphenyl)methyl]-4-piperidinol, hydrochloride,

1-[4-(1H-imidazol-4-yl)-3-butynyl]-4-(phenylmethyl)-piperidine,

1-aminocyclopentane-carboxylic acid,

2-[(2,3-dihydro-1H-inden-2-yl)amino]-acetamide, monohydrochloride,

2-hydroxy-5-[[(pentafluorophenyl)methyl]amino]-benzoic acid,

2-methyl-6-(phenylethynyl)-pyridine,

3-(phosphonomethyl)-L-phenylalanine,

3-[(1E)-2-carboxy-2-phenylethenyl]-4,6-dichloro-1H-indole-2-carboxylic acid,

4,6-dichloro-3-[(E)-(2-oxo-1-phenyl-3-pyrrolidinylidene)methyl]-1H-indole-2-carboxylic acid,

6-chloro-2,3,4,9-tetrahydro-9-methyl-2,3-dioxo-1H-indeno[1,2-b]pyrazine-9-acetic acid,

7-chlorothiokynurenic acid,

8-chloro-2,3-dihydropyridazino[4,5-b]quinoline-1,4-dione 5-oxide salt with 2-hydroxy-N,N,N-trimethyl-ethanaminium,

aptiganel,

besonprodil,

budipine,

conantokin G,

delucemine,

dexanabinol,

felbamate,

fluorofelbamate,

gacyclidine,

glycine,

ipenoxazone,

kaitocephalin,

lanicemine,

licostinel,

midafotel,

milnacipran,

N′-[2-chloro-5-(methylthio)phenyl]-N-methyl-N-[3-(methylthio)phenyl]-guanidine,

N′-[2-chloro-5-(methylthio)phenyl]-N-methyl-N-[3-[(R)-methylsulfinyl]phenyl]-guanidine,

neramexane,

orphenadrine,

remacemide,

topiramate,

α-amino-2-(2-phosphonoethyl)-cyclohexanepropanoic acid, and

α-amino-4-(phosphonomethyl)-benzeneacetic acid

or a pharmaceutically acceptable salt thereof.

Other aspects and embodiments of the invention are more thoroughly detailed below.

Abbreviations and Definitions

The following definitions are provided in order to aid the reader in understanding the detailed description of the present invention.

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfonamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl and trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy. The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N,N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

Another component of the combination of the present invention is a cyclooxygenase-2 selective inhibitor. The terms “cyclooxygenase-2 selective inhibitor”, or “COX-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a COX-2 inhibitor varies depending upon the condition under which the test is performed and on the inhibitors being tested. However, for the purposes of this specification, the selectivity of a COX-2 inhibitor can be measured as a ratio of the in vitro or ex vivo IC₅₀ value for inhibition of COX-1, divided by the IC₅₀ value for inhibition of COX-2 (COX-1 IC₅₀/COX-2 IC₅₀), or as a ratio of the in vivo ED₅₀ value for inhibition of COX-1, divided by the ED₅₀ value for inhibition of COX-2 (COX-1 ED₅₀/COX-2 ED₅₀).

A COX-2 selective inhibitor is any inhibitor for which the ratio of COX-1 IC₅₀ to COX-2 IC₅₀, or the ratio of COX-1 ED₅₀ to COX-2 ED₅₀, is greater than 1. It is preferred that the ratio is greater than 2, more preferably greater than 5, yet more preferably greater than 10, still more preferably greater than 50, and more preferably still greater than 100.

As used herein, the terms “IC₅₀” and “ED₅₀” refer to the concentration of a compound that is required to produce 50% inhibition of cyclooxygenase activity in an in vitro or in vivo test, respectively.

Preferred COX-2 selective inhibitors of the present invention have a COX-2 IC₅₀ of less than about 1 μM, more preferred of less than about 0.5 μM, and even more preferred of less than about 0.2 μM.

Preferred cycloxoygenase-2 selective inhibitors have a COX-1 IC₅₀ of greater than about 1 μM, and more preferably of greater than 20 μM. Such preferred selectivity may indicate an ability to reduce the incidence of common NSAID-induced side effects.

The phrase “combination therapy” (or “co-therapy”) embraces the administration of a COX-2 inhibiting agent and a NMDA antagonist as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients (such as, but not limited to, a NMDA antagonist) and non-drug therapies (such as, but not limited to, surgery or radiation treatment). Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

The phrase “therapeutically effective” is intended to qualify the amount of inhibitors in the therapy. This amount will achieve the goal of treating, preventing or inhibiting neuropathic pain.

“Therapeutic compound” means a compound useful in the treatment, prevention or inhibition of neuropathic pain.

“NMDA receptor antagonist,” and “NMDA antagonist,” are used interchangeably herein and encompass any NMDA receptor antagonist as described in any embodiment herein.

The term “pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, chlorine, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

Combinations and Methods

Among its several embodiments, the present invention provides a composition comprising an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a diarylmethylidenefuran derivative, a 2-phenylaminobenzene acetic acid derivative, a chromene derivative, and parecoxib,

wherein the NMDA antagonist is selected from the group consisting of (−)-6,7-dichloro-1,4-dihydro-5-[3-(methoxymethyl)-5-(3-pyridinyl)-4H-1,2,4-triazol-4-yl]-2,3-quinoxalinedione (UK-315716), (2R,4S)-rel-5,7-dichloro-1,2,3,4-tetrahydro-4-[[(phenylamino)carbonyl]amino]-2-quinolinecarboxylic acid (L 689560), (2R,6S)-1,2,3,4,5,6-hexahydro-3-[(2S)-2-methoxypropyl]-6,11,11-trimethyl-2,6-methano-3-benzazocin-9-ol (BI-II-277-CL), (3E)-2-amino-4-(phosphonomethyl)-3-heptenoic acid (CGP-39653), (3R,4S)-rel-3,4-dihydro-3-[4-hydroxy-4-(phenylmethyl)-1-piperidinyl]-2H-1-benzopyran-4,7-diol (CP-283097), (3S,4aR,6S,8aR)-decahydro-6-(phosphonomethyl)-3-isoquinolinecarboxylic acid (LY-235959), PD-196860, (R)-9-bromo-2,3,6,7-tetrahydro-2,3-dioxo-N-phenyl-1H,5H-pyrido[1,2,3-de]quinoxaline-5-acetamide (SM 31900), (SM-18400), (αR)-α-amino-5-chloro-1-(phosphonomethyl)-1H-benzimidazole-2-propanoic acid (EAB-318), [2-(8,9-dioxo-2,6-diazabicyclo[5.2.0]non-1(7)-en-2-yl)ethyl]-phosphonic acid (EAA-090), [5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride, 1,4-dihydro-6-methyl-5-[(methylamino)methyl]-7-nitro-2,3-quinoxalinedione (PD 165650), 1-[2-(4-hydroxyphenoxy)ethyl]-4-[(4-methylphenyl)methyl]-4-piperidinol, hydrochloride (CO 101244), 1-[4-(1H-imidazol-4-yl)-3-butynyl]-4-(phenylmethyl)-piperidine (PD 188669), 1-aminocyclopentane-carboxylic acid (ACPC), 2-[(2,3-dihydro-1H-inden-2-yl)amino]-acetamide, monohydrochloride (CHF-3381), 2-hydroxy-5-[[(pentafluorophenyl)methyl]amino]-benzoic acid (PBAS), 2-methyl-6-(phenylethynyl)-pyridine (MPEP), 3-(phosphonomethyl)-L-phenylalanine (PD 130527), 3-[(1E)-2-carboxy-2-phenylethenyl]-4,6-dichloro-1H-indole-2-carboxylic acid (MDL 105519), 4,6-dichloro-3-[(E)-(2-oxo-1-phenyl-3-pyrrolidinylidene)methyl]-1H-indole-2-carboxylic acid (GV 196771), 6-chloro-2,3,4,9-tetrahydro-9-methyl-2,3-dioxo-1H-indeno[1,2-b]pyrazine-9-acetic acid (RPR 118723), 7-chlorothiokynurenic acid, 8-chloro-2,3-dihydropyridazino[4,5-b]quinoline-1,4-dione 5-oxide salt with 2-hydroxy-N,N,N-trimethyl-ethanaminium (1,1)(MRZ 2/576), ACEA-1286, aptiganel, AY 12316, besonprodil, budipine, conantokin G, DD-20207, DD-B4, delucemine, dexanabinol, felbamate, fluorofelbamate, gacyclidine, glycine (AZD-4282), GV 117164X, GW-468816, ipenoxazone, kaitocephalin, lanicemine, licostinel, midafotel, milnacipran, N′-[2-chloro-5-(methylthio)phenyl]-N-methyl-N-[3-(methylthio)phenyl]-guanidine (CNS-5161), N′-[2-chloro-5-(methylthio)phenyl]-N-methyl-N-[3-[(R)-methylsulfinyl]phenyl]-guanidine (CNS 5788), NC-1210, neramexane, orphenadrine, remacemide, topiramate, YKP 509, α-amino-2-(2-phosphonoethyl)-cyclohexanepropanoic acid (NPC-12626), and α-amino-4-(phosphonomethyl)-benzeneacetic acid (PD 129653).

In another embodiment, the present invention further provides a combination therapy method for the treatment, prevention, or inhibition of neuropathic pain in a mammal in need thereof, comprising administering to the mammal an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a diarylmethylidenefuran derivative, a 2-phenylaminobenzene acetic acid derivative, a chromene derivative, and parecoxib,

wherein the NMDA antagonist is selected from the group consisting of (−)-6,7-dichloro-1,4-dihydro-5-[3-(methoxymethyl)-5-(3-pyridinyl)-4H-1,2,4-triazol-4-yl]-2,3-quinoxalinedione (UK-315716), (2R,4S)-rel-5,7-dichloro-1,2,3,4-tetrahydro-4-[[(phenylamino)carbonyl]amino]-2-quinolinecarboxylic acid (L 689560), (2R,6S)-1,2,3,4,5,6-hexahydro-3-[(2S)-2-methoxypropyl]-6,11,11-trimethyl-2,6-methano-3-benzazocin-9-ol (BI-II-277-CL), (3E)-2-amino-4-(phosphonomethyl)-3-heptenoic acid (CGP-39653), (3R,4S)-rel-3,4-dihydro-3-[4-hydroxy-4-(phenylmethyl)-1-piperidinyl]-2H-1-benzopyran-4,7-diol (CP-283097), (3S,4aR,6S,8aR)-decahydro-6-(phosphonomethyl)-3-isoquinolinecarboxylic acid (LY-235959), PD-196860, (R)-9-bromo-2,3,6,7-tetrahydro-2,3-dioxo-N-phenyl-1H,5H-pyrido[1,2,3-de]quinoxaline-5-acetamide (SM 31900), (SM-18400), (αR)-α-amino-5-chloro-1-(phosphonomethyl)-1H-benzimidazole-2-propanoic acid (EAB-318), [2-(8,9-dioxo-2,6-diazabicyclo[5.2.0]non-1(7)-en-2-yl)ethyl]-phosphonic acid (EAA-090), [5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride, 1,4-dihydro-6-methyl-5-[(methylamino)methyl]-7-nitro-2,3-quinoxalinedione (PD 165650), 1-[2-(4-hydroxyphenoxy)ethyl]-4-[(4-methylphenyl)methyl]-4-piperidinol, hydrochloride (Co 101244), 1-[4-(1H-imidazol-4-yl)-3-butynyl]-4-(phenylmethyl)-piperidine (PD 188669), 1-aminocyclopentane-carboxylic acid (ACPC), 2-[(2,3-dihydro-1H-inden-2-yl)amino]-acetamide, monohydrochloride (CHF-3381), 2-hydroxy-5-[[(pentafluorophenyl)methyl]amino]-benzoic acid (PBAS), 2-methyl-6-(phenylethynyl)-pyridine (MPEP), 3-(phosphonomethyl)-L-phenylalanine (PD 130527), 3-[(1E)-2-carboxy-2-phenylethenyl]-4,6-dichloro-1H-indole-2-carboxylic acid (MDL 105519), 4,6-dichloro-3-[(E)-(2-oxo-1-phenyl-3-pyrrolidinylidene)methyl]-1H-indole-2-carboxylic acid (GV 196771), 6-chloro-2,3,4,9-tetrahydro-9-methyl-2,3-dioxo-1H-indeno[1,2-b]pyrazine-9-acetic acid (RPR 118723), 7-chlorothiokynurenic acid, 8-chloro-2,3-dihydropyridazino[4,5-b]quinoline-1,4-dione 5-oxide salt with 2-hydroxy-N,N,N-trimethyl-ethanaminium (1,1)(MRZ 2/576), ACEA-1286, aptiganel, AY 12316, besonprodil, budipine, conantokin G, DD-20207, DD-B4, delucemine, dexanabinol, felbamate, fluorofelbamate, gacyclidine, glycine (AZD-4282), GV 117164X, GW-468816, ipenoxazone, kaitocephalin, lanicemine, licostinel, midafotel, milnacipran, N′-[2-chloro-5-(methylthio)phenyl]-N-methyl-N-[3-(methylthio)phenyl]-guanidine (CNS-5161), N′-[2-chloro-5-(methylthio)phenyl]-N-methyl-N-[3-[(R)-methylsulfinyl]phenyl]-guanidine (CNS 5788), NC-1210, neramexane, orphenadrine, remacemide, topiramate, YKP 509, α-amino-2-(2-phosphonoethyl)-cyclohexanepropanoic acid (NPC-12626), and α-amino-4-(phosphonomethyl)-benzeneacetic acid (PD 129653).

In a further embodiment, the present invention provides a pharmaceutical composition for the treatment, prevention, or inhibition of neuropathic pain comprising an amount of a COX-2 selective inhibitor source and an amount of a NMDA antagonist and a pharmaceutically-acceptable excipient,

wherein the COX-2 selective inhibitor source is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a diarylmethylidenefuran derivative, a 2-phenylaminobenzene acetic acid derivative, a chromene derivative, and parecoxib,

wherein the NMDA antagonist is selected from the group consisting of (−)-6,7-dichloro-1,4-dihydro-5-[3-(methoxymethyl)-5-(3-pyridinyl)-4H-1,2,4-triazol-4-yl]-2,3-quinoxalinedione (UK-315716), (2R,4S)-rel-5,7-dichloro-1,2,3,4-tetrahydro-4-[[(phenylamino)carbonyl]amino]-2-quinolinecarboxylic acid (L 689560), (2R,6S)-1,2,3,4,5,6-hexahydro-3-[(2S)-2-methoxypropyl]-6,11,11-trimethyl-2,6-methano-3-benzazocin-9-ol (BI-II-277-CL), (3E)-2-amino-4-(phosphonomethyl)-3-heptenoic acid (CGP-39653), (3R,4S)-rel-3,4-dihydro-3-[4-hydroxy-4-(phenylmethyl)-1-piperidinyl]-2H-1-benzopyran-4,7-diol (CP-283097), (3S,4aR,6S,8aR)-decahydro-6-(phosphonomethyl)-3-isoquinolinecarboxylic acid (LY-235959), PD-196860, (R)-9-bromo-2,3,6,7-tetrahydro-2,3-dioxo-N-phenyl-1H,5H-pyrido[1,2,3-de]quinoxaline-5-acetamide (SM 31900), (SM-18400), (αR)-α-amino-5-chloro-1-(phosphonomethyl)-1H-benzimidazole-2-propanoic acid (EAB-318), [2-(8,9-dioxo-2,6-diazabicyclo[5.2.0]non-1(7)-en-2-yl)ethyl]-phosphonic acid (EAA-090), [5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride, 1,4-dihydro-6-methyl-5-[(methylamino)methyl]-7-nitro-2,3-quinoxalinedione (PD 165650), 1-[2-(4-hydroxyphenoxy)ethyl]-4-[(4-methylphenyl)methyl]-4-piperidinol, hydrochloride (CO 101244), 1-[4-(1H-imidazol-4-yl)-3-butynyl]-4-(phenylmethyl)-piperidine (PD 188669), 1-aminocyclopentane-carboxylic acid (ACPC), 2-[(2,3-dihydro-1H-inden-2-yl)amino]-acetamide, monohydrochloride (CHF-3381), 2-hydroxy-5-[[(pentafluorophenyl)methyl]amino]-benzoic acid (PBAS), 2-methyl-6-(phenylethynyl)-pyridine (MPEP), 3-(phosphonomethyl)-L-phenylalanine (PD 130527), 3-[(1E)-2-carboxy-2-phenylethenyl]-4,6-dichloro-1H-indole-2-carboxylic acid (MDL 105519), 4,6-dichloro-3-[(E)-(2-oxo-1-phenyl-3-pyrrolidinylidene)methyl]-1H-indole-2-carboxylic acid (GV 196771), 6-chloro-2,3,4,9-tetrahydro-9-methyl-2,3-dioxo-1H-indeno[1,2-b]pyrazine-9-acetic acid (RPR 118723), 7-chlorothiokynurenic acid, 8-chloro-2,3-dihydropyridazino[4,5-b]quinoline-1,4-dione 5-oxide salt with 2-hydroxy-N,N,N-trimethyl-ethanaminium (1,1)(MRZ 2/576), ACEA-1286, aptiganel, AY 12316, besonprodil, budipine, conantokin G, DD-20207, DD-B4, delucemine, dexanabinol, felbamate, fluorofelbamate, gacyclidine, glycine (AZD-4282), GV 117164X, GW-468816, ipenoxazone, kaitocephalin, lanicemine, licostinel, midafotel, milnacipran, N′-[2-chloro-5-(methylthio)phenyl]-N-methyl-N-[3-(methylthio)phenyl]-guanidine (CNS-5161), N′-[2-chloro-5-(methylthio)phenyl]-N-methyl-N-[3-[(R)-methylsulfinyl]phenyl]-guanidine (CNS 5788), NC-1210, neramexane, orphenadrine, remacemide, topiramate, YKP 509, α-amino-2-(2-phosphonoethyl)-cyclohexanepropanoic acid (NPC-12626), and α-amino-4-(phosphonomethyl)-benzeneacetic acid (PD 129653).

In yet another embodiment, the present invention provides a kit that is suitable for use in the treatment, prevention or inhibition of neuropathic pain, wherein the kit comprises a first dosage form comprising a COX-2 selective inhibitor source and a second dosage form comprising a NMDA antagonist, in quantities which comprise a therapeutically effective amount of the compounds for the treatment, prevention or inhibition of neuropathic pain,

wherein the COX-2 selective inhibitor source is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a diarylmethylidenefuran derivative, a 2-phenylaminobenzene acetic acid derivative, a chromene derivative, and parecoxib,

wherein the NMDA antagonist is selected from the group consisting of (−)-6,7-dichloro-1,4-dihydro-5-[3-(methoxymethyl)-5-(3-pyridinyl)-4H-1,2,4-triazol-4-yl]-2,3-quinoxalinedione (UK-315716), (2R,4S)-rel-5,7-dichloro-1,2,3,4-tetrahydro-4-[[(phenylamino)carbonyl]amino]-2-quinolinecarboxylic acid (L 689560), (2R,6S)-1,2,3,4,5,6-hexahydro-3-[(2S)-2-methoxypropyl]-6,11,11-trimethyl-2,6-methano-3-benzazocin-9-ol (BI-II-277-CL), (3E)-2-amino-4-(phosphonomethyl)-3-heptenoic acid (CGP-39653), (3R,4S)-rel-3,4-dihydro-3-[4-hydroxy-4-(phenylmethyl)-1-piperidinyl]-2H-1-benzopyran-4,7-diol (CP-283097), (3S,4aR,6S,8aR)-decahydro-6-(phosphonomethyl)-3-isoquinolinecarboxylic acid (LY-235959), PD-196860, (R)-9-bromo-2,3,6,7-tetrahydro-2,3-dioxo-N-phenyl-1H,5H-pyrido[1,2,3-de]quinoxaline-5-acetamide (SM 31900), (SM-18400), (αR)-α-amino-5-chloro-1-(phosphonomethyl)-1H-benzimidazole-2-propanoic acid (EAB-318), [2-(8,9-dioxo-2,6-diazabicyclo[5.2.0]non-1(7)-en-2-yl)ethyl]-phosphonic acid (EAA-090), [5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride, 1,4-dihydro-6-methyl-5-[(methylamino)methyl]-7-nitro-2,3-quinoxalinedione (PD 165650), 1-[2-(4-hydroxyphenoxy)ethyl]-4-[(4-methylphenyl)methyl]-4-piperidinol, hydrochloride (CO 101244), 1-[4-(1H-imidazol-4-yl)-3-butynyl]-4-(phenylmethyl)-piperidine (PD 188669), 1-aminocyclopentane-carboxylic acid (ACPC), 2-[(2,3-dihydro-1H-inden-2-yl)amino]-acetamide, monohydrochloride (CHF-3381), 2-hydroxy-5-[[(pentafluorophenyl)methyl]amino]-benzoic acid (PBAS), 2-methyl-6-(phenylethynyl)-pyridine (MPEP), 3-(phosphonomethyl)-L-phenylalanine (PD 130527), 3-[(1E)-2-carboxy-2-phenylethenyl]-4,6-dichloro-1H-indole-2-carboxylic acid (MDL 105519), 4,6-dichloro-3-[(E)-(2-oxo-1-phenyl-3-pyrrolidinylidene)methyl]-1H-indole-2-carboxylic acid (GV 196771), 6-chloro-2,3,4,9-tetrahydro-9-methyl-2,3-dioxo-1H-indeno[1,2-b]pyrazine-9-acetic acid (RPR 118723), 7-chlorothiokynurenic acid, 8-chloro-2,3-dihydropyridazino[4,5-b]quinoline-1,4-dione 5-oxide salt with 2-hydroxy-N,N,N-trimethyl-ethanaminium (1,1)(MRZ 2/576), ACEA-1286, aptiganel, AY 12316, besonprodil, budipine, conantokin G, DD-20207, DD-B4, delucemine, dexanabinol, felbamate, fluorofelbamate, gacyclidine, glycine (AZD-4282), GV 117164X, GW-468816, ipenoxazone, kaitocephalin, lanicemine, licostinel, midafotel, milnacipran, N′-[2-chloro-5-(methylthio)phenyl]-N-methyl-N-[3-(methylthio)phenyl]-guanidine (CNS-5161), N′-[2-chloro-5-(methylthio)phenyl]-N-methyl-N-[3-[(R)-methylsulfinyl]phenyl]-guanidine (CNS 5788), NC-1210, neramexane, orphenadrine, remacemide, topiramate, YKP 509, α-amino-2-(2-phosphonoethyl)-cyclohexanepropanoic acid (NPC-12626), and α-amino-4-(phosphonomethyl)-benzeneacetic acid (PD 129653).

Among further embodiments, the present invention provides a composition comprising an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a 2-phenylaminobenzene acetic acid derivative, a chromene derivative, and parecoxib,

wherein the NMDA antagonist is selected from the group consisting of amantidine, dextromethorphan, ketamine, memantine, and traxoprodil.

In a still further embodiment, the present invention provides a combination therapy method for the treatment, prevention, or inhibition of neuropathic pain in a mammal in need thereof, comprising administering to the mammal an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a 2-phenylaminobenzene acetic acid derivative, a chromene derivative, and parecoxib,

wherein the NMDA antagonist is selected from the group consisting of amantidine, dextromethorphan, ketamine, memantine, and traxoprodil.

In another embodiment, the present invention provides pharmaceutical composition for the treatment, prevention, or inhibition of neuropathic pain comprising an amount of a COX-2 selective inhibitor source and an amount of a NMDA antagonist and a pharmaceutically-acceptable excipient,

wherein the COX-2 selective inhibitor source is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a 2-phenylaminobenzene acetic acid derivative, a chromene derivative, and parecoxib,

wherein the NMDA antagonist is selected from the group consisting of amantidine, dextromethorphan, ketamine, memantine, and traxoprodil.

In still another embodiment, the present invention provides a kit that is suitable for use in the treatment, prevention or inhibition of neuropathic pain, wherein the kit comprises a first dosage form comprising a COX-2 selective inhibitor source and a second dosage form comprising a NMDA antagonist, in quantities which comprise a therapeutically effective amount of the compounds for the treatment, prevention or inhibition of neuropathic pain,

wherein the COX-2 selective inhibitor source is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a 2-phenylaminobenzene acetic acid derivative, a chromene derivative, and parecoxib,

wherein the NMDA antagonist is selected from the group consisting of amantidine, dextromethorphan, ketamine, memantine, and traxoprodil.

Among yet other embodiments, the present invention provides a composition comprising an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide,

wherein the NMDA antagonist is selected from the group consisting of amantidine, dextromethorphan, ketamine, and memantine.

Other embodiments of the present invention include a combination therapy method for the treatment, prevention, or inhibition of neuropathic pain in a mammal in need thereof, comprising administering to the mammal an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide,

wherein the NMDA antagonist is selected from the group consisting of amantidine, dextromethorphan, ketamine, and memantine.

In an even further embodiment, the present invention provides a pharmaceutical composition for the treatment, prevention, or inhibition of neuropathic pain comprising an amount of a COX-2 selective inhibitor source and an amount of a NMDA antagonist and a pharmaceutically-acceptable excipient,

wherein the COX-2 selective inhibitor source is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide,

wherein the NMDA antagonist is selected from the group consisting of amantidine, dextromethorphan, ketamine, and memantine.

A still further embodiment of the present invention provides a kit that is suitable for use in the treatment, prevention or inhibition of neuropathic pain, wherein the kit comprises a first dosage form comprising a COX-2 selective inhibitor source and a second dosage form comprising a NMDA antagonist, in quantities which comprise a therapeutically effective amount of the compounds for the treatment, prevention or inhibition of neuropathic pain,

wherein the COX-2 selective inhibitor source is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide,

wherein the NMDA antagonist is selected from the group consisting of amantidine, dextromethorphan, ketamine, and memantine.

Still another embodiment of the present invention provides a composition comprising an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is a diarylmethylidenefuran derivative,

wherein the NMDA antagonist is selected from the group consisting of amantidine, memantine, and traxoprodil.

A further embodiment of the present invention provides a combination therapy method for the treatment, prevention, or inhibition of neuropathic pain in a mammal in need thereof, comprising administering to the mammal an amount of a COX-2 selective inhibitor source and an amount of a NMDA antagonist wherein the amount of the COX-2 selective inhibitor source and the amount of the NMDA antagonist together comprise a therapeutically effective amount for the treatment, prevention, or inhibition of neuropathic pain,

wherein the COX-2 selective inhibitor source is a diarylmethylidenefuran derivative,

wherein the NMDA antagonist is selected from the group consisting of amantidine, memantine, and traxoprodil.

Yet another embodiment of the present invention provides a pharmaceutical composition for the treatment, prevention, or inhibition of neuropathic pain comprising an amount of a COX-2 selective inhibitor source and an amount of a NMDA antagonist and a pharmaceutically-acceptable excipient,

wherein the COX-2 selective inhibitor source is a diarylmethylidenefuran derivative,

wherein the NMDA antagonist is selected from the group consisting of amantidine, memantine, and traxoprodil.

A further embodiment of the present invention provides a kit that is suitable for use in the treatment, prevention or inhibition of neuropathic pain, wherein the kit comprises a first dosage form comprising a COX-2 selective inhibitor source and a second dosage form comprising a NMDA antagonist, in quantities which comprise a therapeutically effective amount of the compounds for the treatment, prevention or inhibition of neuropathic pain,

wherein the COX-2 selective inhibitor source is a diarylmethylidenefuran derivative,

wherein the NMDA antagonist is selected from the group consisting of amantidine, memantine, and traxoprodil.

The methods and compositions of the present invention provide one or more benefits. Combinations of COX-2 inhibitors and NMDA antagonists are useful in treating, preventing or inhibiting neuropathic pain. Preferably, the COX-2 inhibitors and the NMDA antagonists of the present invention are administered in combination at a low dose, that is, at a dose lower than has been conventionally used in clinical situations.

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Alternatively, the methods and combination of the present invention can also maximize the therapeutic effect at higher doses.

When administered as a combination, the therapeutic agents can be formulated as separate compositions that are given at the same time or different times, or the therapeutic agents can be given as a single composition.

There are many uses for the present inventive combination. For example, NMDA antagonists and COX-2 selective inhibiting agents (or prodrugs thereof) are each believed to be effective analgesic agents. The present inventive combination will allow the subject to be administered a NMDA antagonist and a COX-2 inhibitor at a therapeutically effective dose yet experience reduced or fewer symptoms of side effects. A further use and advantage is that the present inventive combination will allow therapeutically effective individual dose levels of the NMDA antagonist and the COX-2 inhibitor that are lower than the dose levels of each inhibitor when administered to the patient as a monotherapy.

Inhibitors of the cyclooxygenase pathway in the metabolism of arachidonic acid used in the treatment, prevention or reduction of neuropathic pain may inhibit enzyme activity through a variety of mechanisms. By way of example, the cyclooxygenase-2 inhibitors used in the methods described herein may block the enzyme activity directly by acting as a substrate for the enzyme. The use of a COX-2 selective inhibiting agent is highly advantageous in that they minimize the gastric side effects that can occur with non-selective non-steroidal anti-inflammatory drugs (NSAIDs), especially where prolonged treatment is expected.

Besides being useful for human treatment, these methods are also useful for veterinary treatment of companion animals, exotic animals and farm animals, including mammals, rodents, avians, and the like. More preferred animals include horses, dogs, and cats.

Preferred COX-2 selective inhibitors that may be used in the present invention include, but are not limited to:

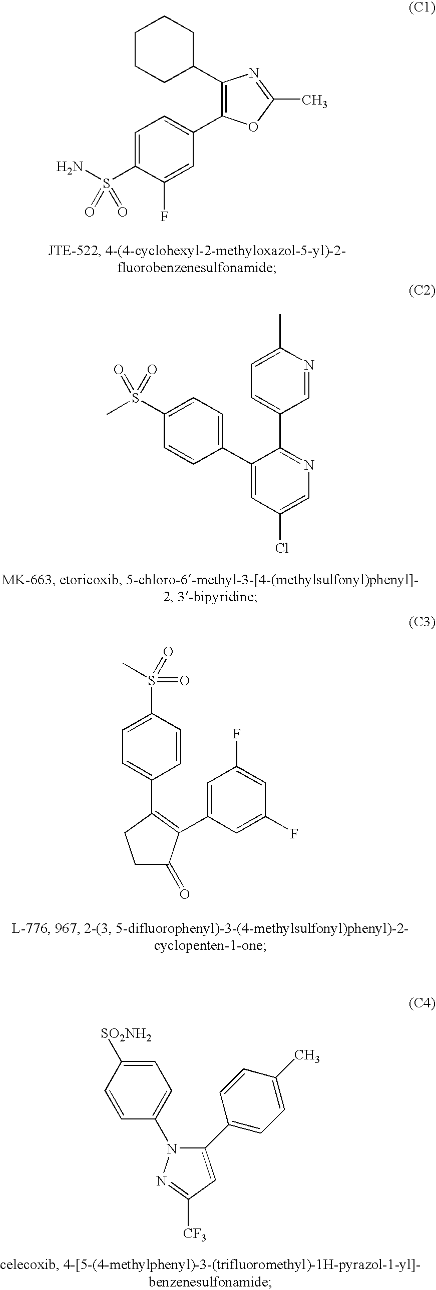

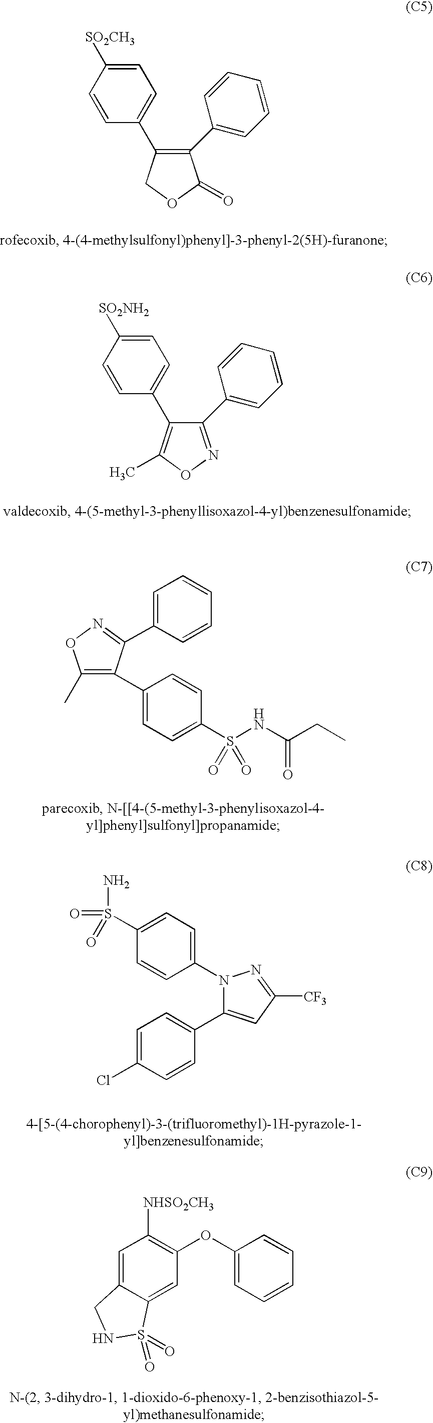

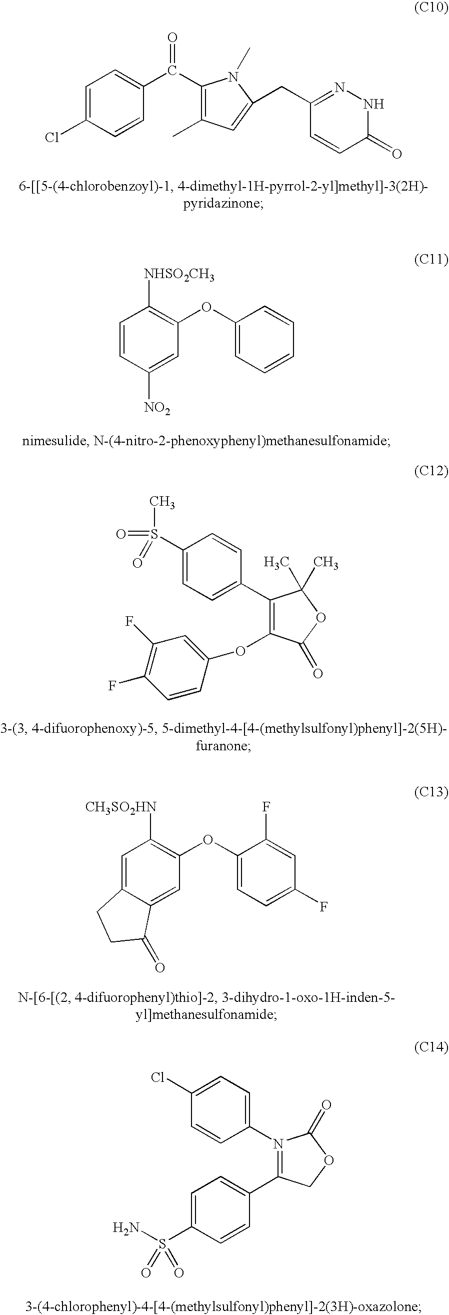

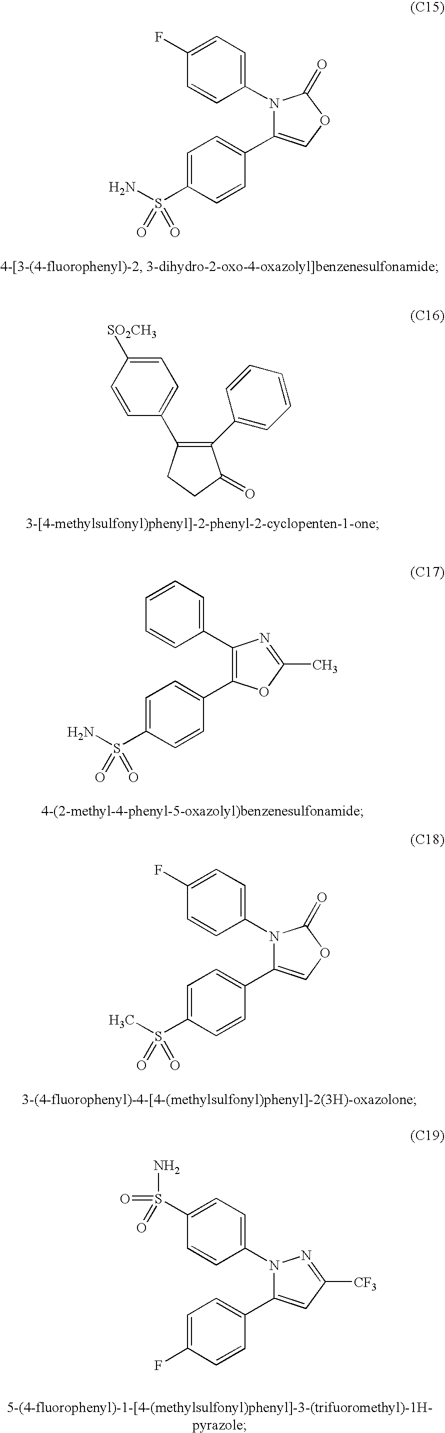

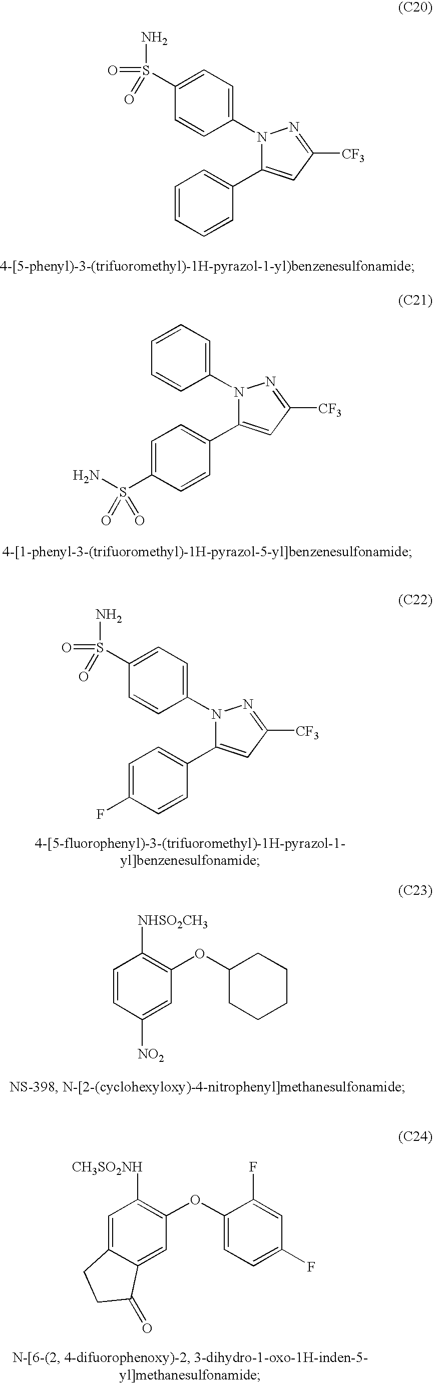

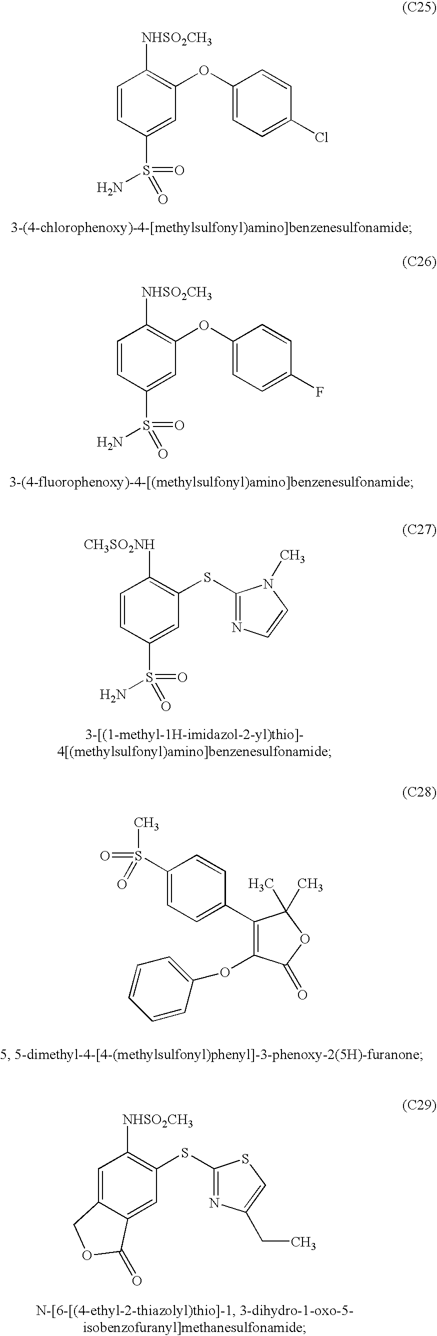

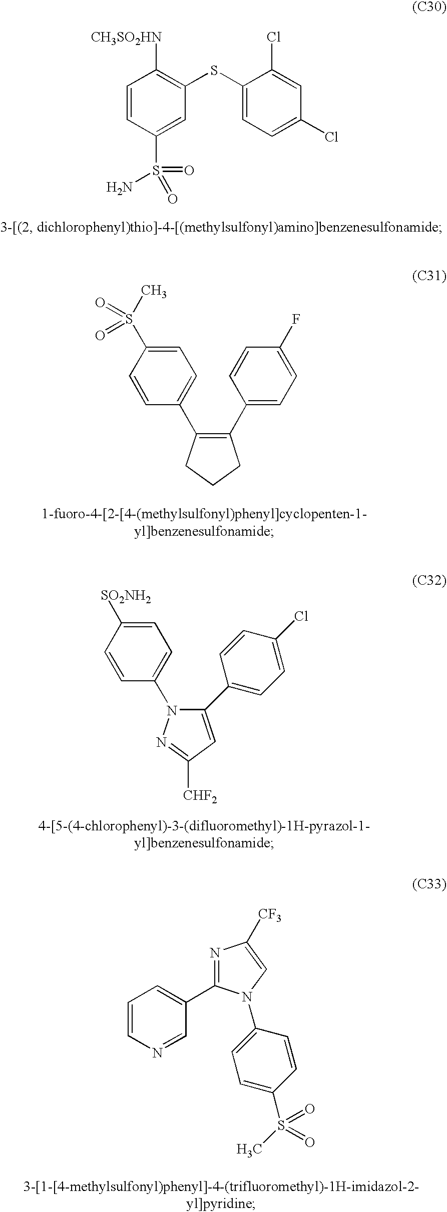

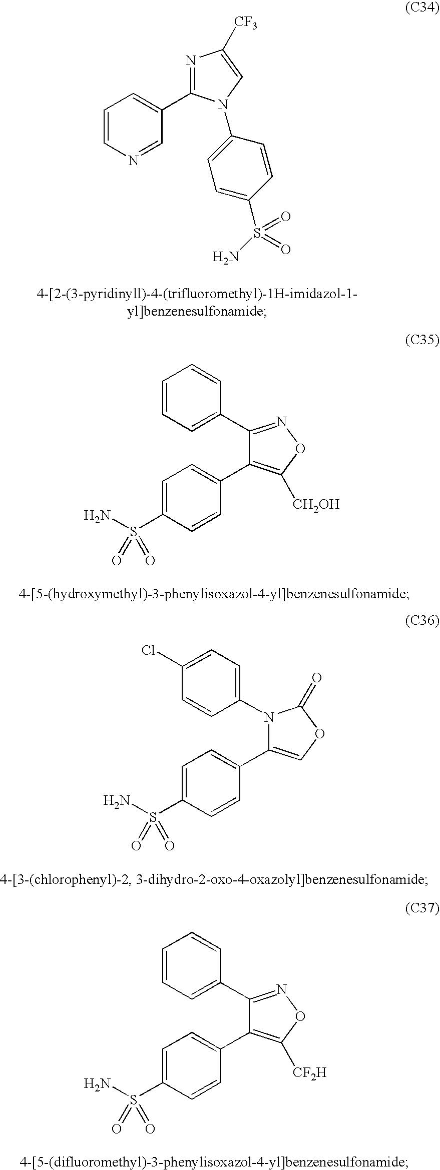

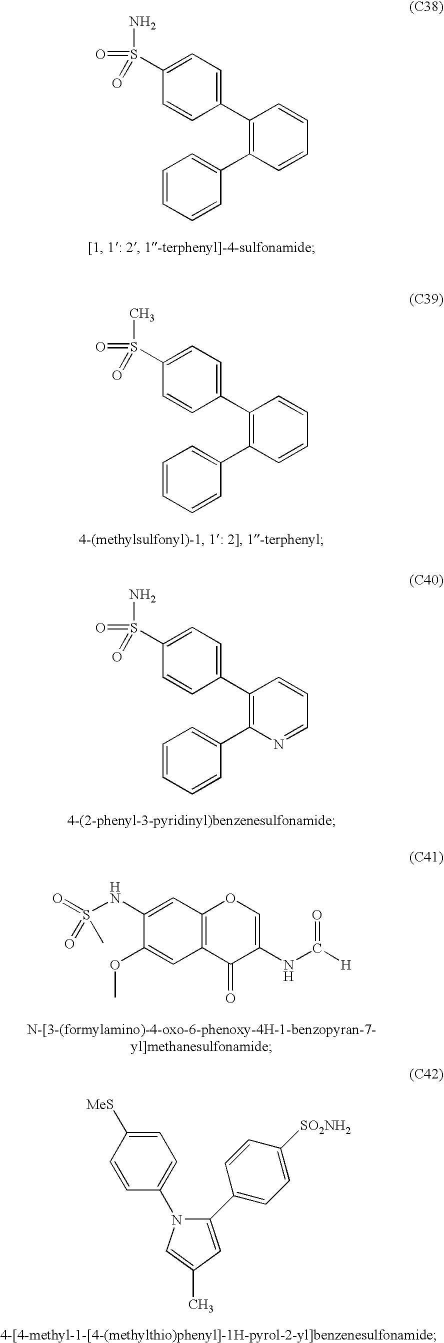

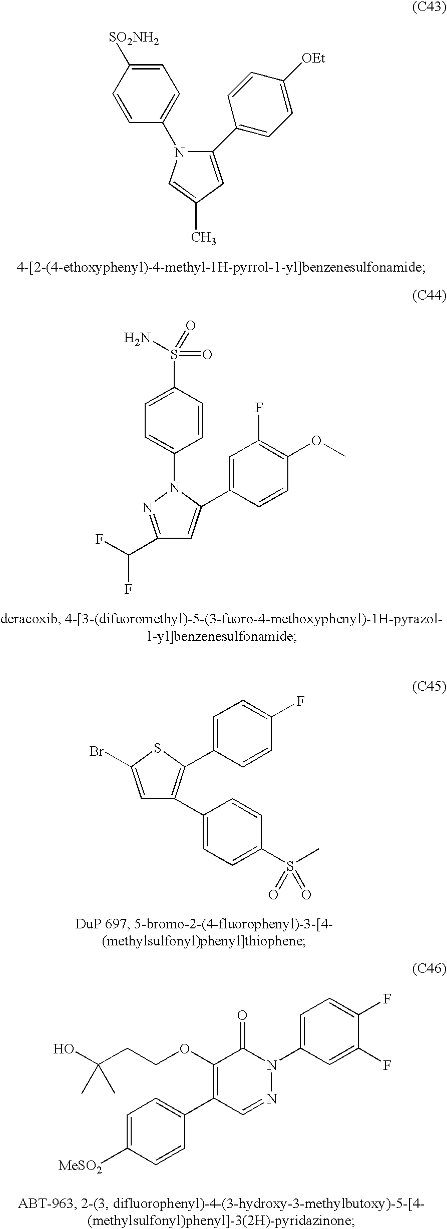

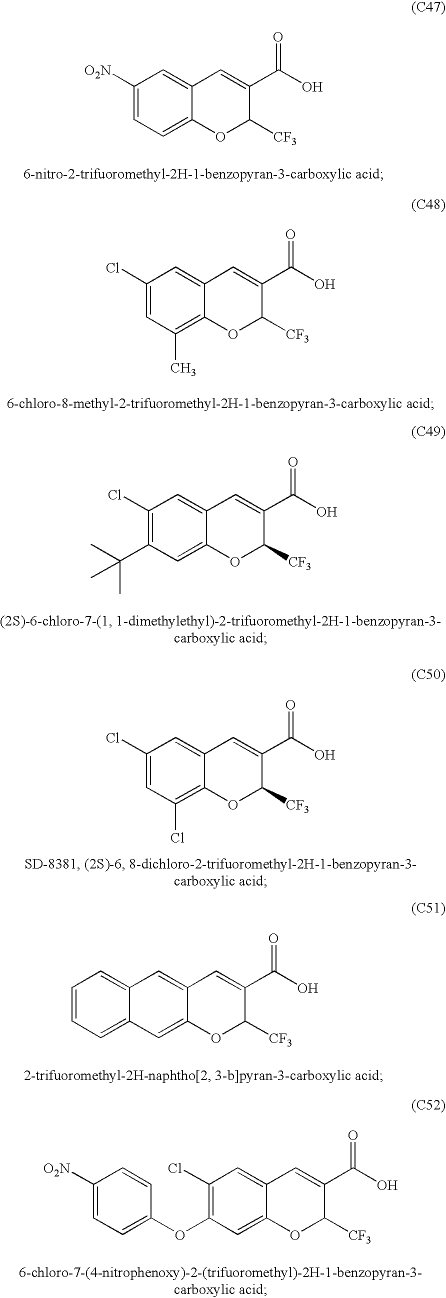

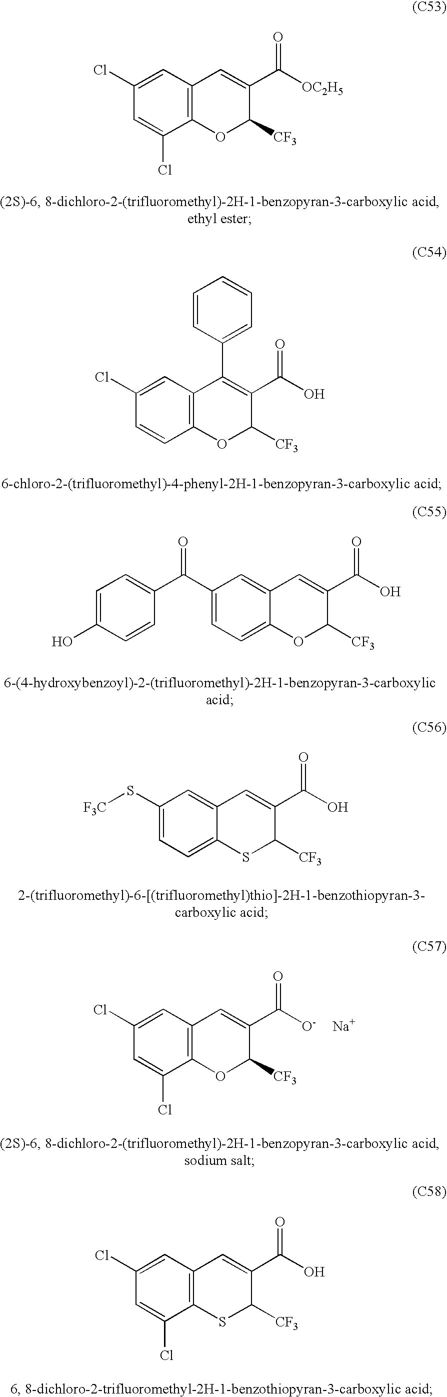

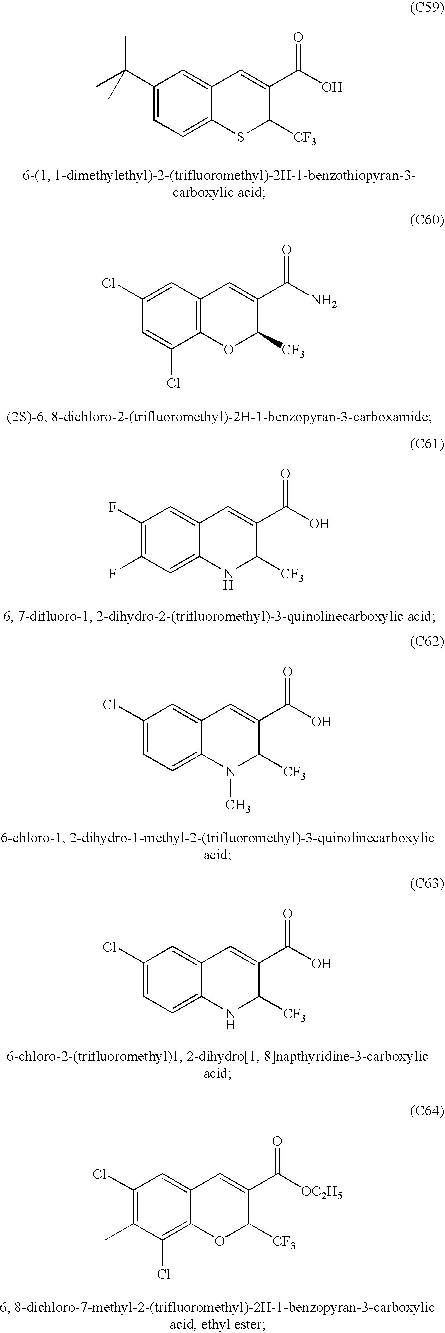

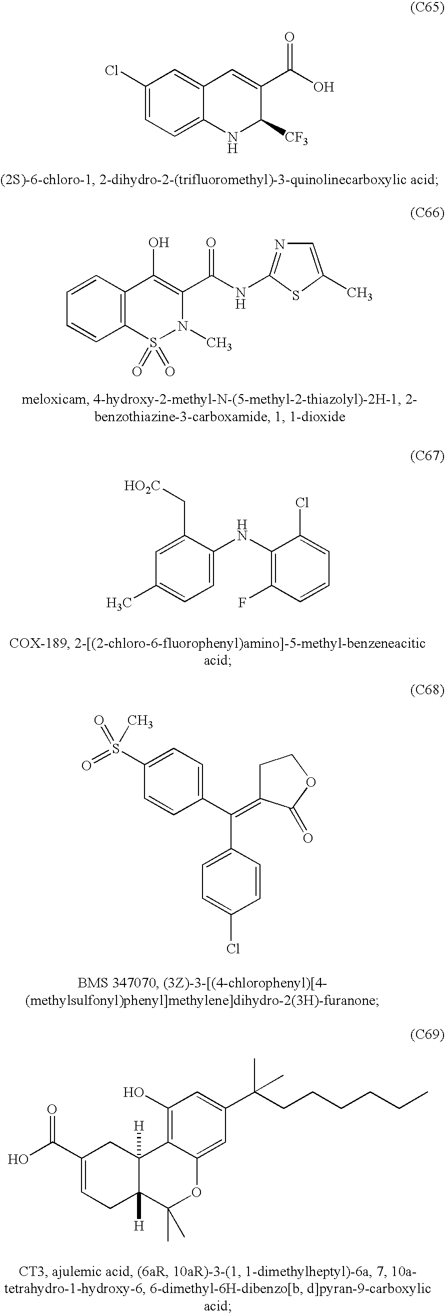

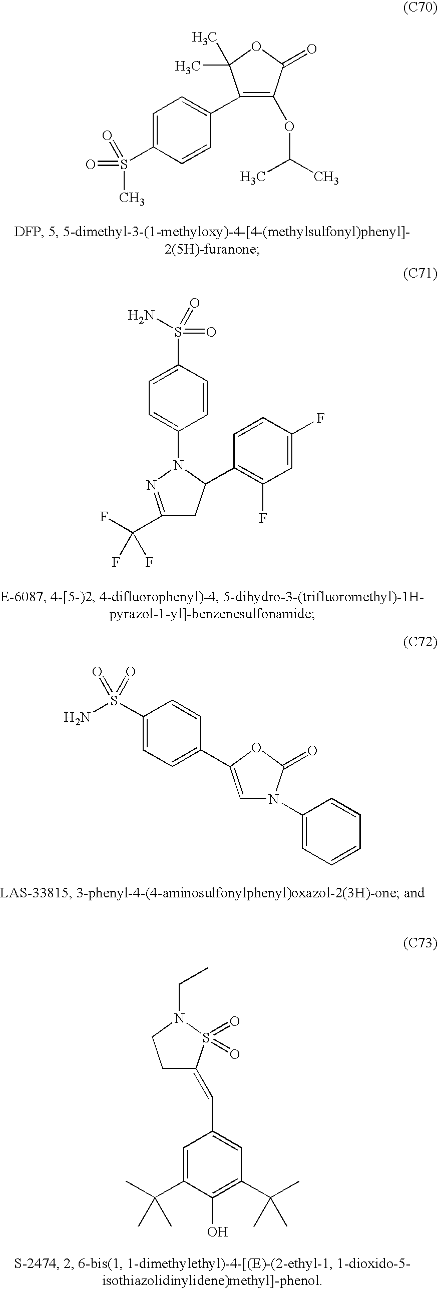

The CAS reference numbers for nonlimiting examples of COX-2 inhibitors are identified in Table No. 1 below.

TABLE No. 1
COX-2 Inhibitor's CAS Reference Numbers

| Compound | |
|---|---|
| Number | CAS Reference Number |
| C1 | 180200-68-4 |
| C2 | 202409-33-4 |
| C3 | 212126-32-4 |
| C4 | 169590-42-5 |
| C5 | 162011-90-7 |
| C6 | 181695-72-7 |
| C7 | 198470-84-7 |
| C8 | 170569-86-5 |
| C9 | 187845-71-2 |
| C10 | 179382-91-3 |
| C11 | 51803-78-2 |
| C12 | 189954-13-0 |
| C13 | 158205-05-1 |
| C14 | 197239-99-9 |
| C15 | 197240-09-8 |
| C16 | 226703-01-1 |
| C17 | 93014-16-5 |
| C18 | 197239-97-7 |
| C19 | 162054-19-5 |
| C20 | 170569-87-6 |
| C21 | 279221-13-5 |
| C22 | 170572-13-1 |
| C23 | 123653-11-2 |
| C24 | 80937-31-1 |
| C25 | 279221-14-6 |
| C26 | 279221-15-7 |
| C27 | 187846-16-8 |
| C28 | 189954-16-3 |
| C29 | 181485-41-6 |
| C30 | 187845-80-3 |
| C31 | 158959-32-1 |
| C32 | 170570-29-3 |
| C33 | 177660-77-4 |
| C34 | 177660-95-6 |
| C35 | 181695-81-8 |
| C36 | 197240-14-5 |
| C37 | 181696-33-3 |
| C38 | 178816-94-9 |
| C39 | 178816-61-0 |
| C40 | 279221-17-9 |
| C41 | 123663-49-0 |
| C42 | 197905-01-4 |
| C43 | 197904-84-0 |
| C44 | 169590-41-4 |
| C45 | 88149-94-4 |
| C46 | 266320-83-6 |
| C47 | 215122-43-3 |
| C48 | 215122-44-4 |
| C49 | 215122-74-0 |
| C50 | 215123-80-1 |
| C51 | 215122-70-6 |
| C52 | 264878-87-7 |
| C53 | 279221-12-4 |
| C54 | 215123-48-1 |
| C55 | 215123-03-8 |
| C56 | 215123-60-7 |
| C57 | 279221-18-0 |
| C58 | 215123-61-8 |
| C59 | 215123-52-7 |
| C60 | 279221-19-1 |
| C61 | 215123-64-1 |
| C62 | 215123-70-9 |
| C63 | 215123-79-8 |
| C64 | 215123-91-4 |
| C65 | 215123-77-6 |
| C66 | 71125-38-7 |
| C67 | 220991-20-8 |
| C68 | 197438-41-8 |
| C69 | 137945-48-3 |
| C70 | 189954-66-3 |
| C71 | 251442-94-1 |
| C73 | 158089-95-3 |

More preferably, the COX-2 inhibitor sources that may be used in the present invention include, but are not limited to celecoxib, deracoxib, valdecoxib, chromene COX-2 inhibitors, parecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide and 2-[(2-chloro-6-fluorophenyl)amino]-5-methyl-benzeneacetic acid, (3Z)-3-[(4-chlorophenyl)[4-(methylsulfonyl)phenyl]methylene]dihydro-2(3H)-furanone, and diarylmethylidenefuran derivative COX-2 inhibitors.

The compound SD-8381, shown as structure (C50), is a preferred chromene-type COX-2 selective inhibitor. The sodium salt form of the compound is preferred. Further information about SD-8381 can be found in U.S. Pat. No. 6,034,256.

Also included within the scope of the present invention are compounds that act as prodrugs of COX-2 selective inhibitors. As used herein in reference to COX-2 selective inhibitors, the term “prodrug” refers to a chemical compound that can be converted into an active COX-2 selective inhibitor by metabolic or simple chemical processes within the body of the subject. One example of a prodrug for a COX-2 selective inhibitor is parecoxib, which is a therapeutically effective prodrug of the tricyclic cyclooxygenase-2 selective inhibitor valdecoxib. An example of a preferred COX-2 selective inhibitor prodrug is parecoxib sodium. A class of prodrugs of COX-2 inhibitors is described in U.S. Pat. No. 5,932,598.

A class of chromene COX-2 selective inhibiting agents useful in the methods and combinations of the present invention includes compounds of Formula (2),

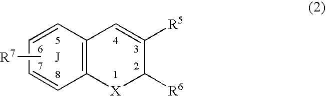

wherein X is selected from the group consisting of 0 or S or NR^{a};

wherein R^{a} is alkyl;

wherein R⁵ is selected from the group consisting of carboxyl, aminocarbonyl, alkylsulfonylaminocarbonyl and alkoxycarbonyl;

wherein R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

wherein R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring J forms a naphthyl radical;

or an isomer or pharmaceutically acceptable salt thereof.

A preferred class of compounds within Formula (2) includes compounds wherein X is oxygen;

R⁵ is selected from the group consisting of carboxyl, lower alkyl, lower aralkyl and lower alkoxycarbonyl;

R⁶ is selected from the group consisting of lower haloalkyl, lower cycloalkyl and phenyl; and

R⁷ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or wherein R⁷ together with ring J forms a naphthyl radical;

or an isomer or pharmaceutically acceptable salt thereof.

A more preferred class of compounds within Formula (2) includes compounds wherein R⁵ is carboxyl;

R⁶ is lower haloalkyl; and

R⁷ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R⁷ together with ring J forms a naphthyl radical;

or an isomer or pharmaceutically acceptable salt thereof.

A still more preferred class of compounds within Formula (2) includes compounds wherein R⁶ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R⁷ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or wherein R⁷ together with ring J forms a naphthyl radical;

or an isomer or pharmaceutically acceptable salt thereof.

An even more preferred class of compounds within Formula (2) includes compounds wherein R⁶ is selected from the group consisting of trifluoromethyl and pentafluorethyl; and

R⁷ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R⁷ together with ring J forms a naphthyl radical;

or an isomer or pharmaceutically acceptable salt thereof.

Another class of chromene COX-2 selective inhibiting agents useful in the methods and combinations of the present invention includes compounds of Formula (3),

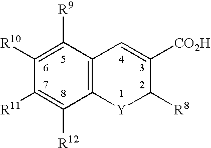

wherein Y is selected from the group consisting of O and S;

R⁸ is lower haloalkyl;

R⁹ is selected from the group consisting of hydrido, and halo;

wherein R¹⁰ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6-membered nitrogen-containing heterocyclosulfonyl;

R¹¹ is selected from the group consisting of hydrido, lower alkyl, halo, lower alkoxy, and aryl; and

R¹² is selected from the group consisting of the group consisting of hydrido, halo, lower alkyl, lower alkoxy, and aryl;

or an isomer or pharmaceutically acceptable salt thereof.

A preferred class of compounds within Formula (3) includes compounds wherein R⁸ is selected from the group consisting of trifluoromethyl and pentafluoroethyl;

R⁹ is selected from the group consisting of hydrido, chloro, and fluoro;

R¹⁰ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R¹¹ is selected from the group consisting of hydrido, methyl, ethyl, isopropyl, tert-butyl, chloro, methoxy, diethylamino, and phenyl; and

R¹² is selected from the group consisting of hydrido, chloro, bromo, fluoro, methyl, ethyl, tert-butyl, methoxy, and phenyl;

or an isomer or pharmaceutically acceptable salt thereof.

A further class of COX-2 selective inhibiting agents useful in the methods and combinations of the present invention includes 5-alkyl-2-arylaminophenylacetic acid compounds of Formula (4)

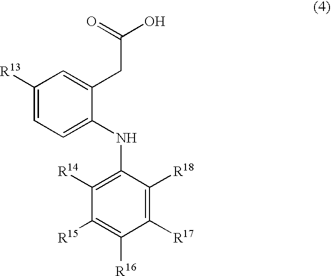

or a pharmaceutically acceptable salt thereof, or a pharmaceutically acceptable prodrug ester thereof, wherein:

R¹³ is methyl or ethyl;

R¹⁴ is chloro or fluoro;

R¹⁵ is hydrogen or fluoro;

R¹⁶ is hydrogen, fluoro, chloro, methyl, ethyl, methoxy, ethoxy or hydroxy;

R¹⁷ is hydrogen or fluoro; and

R¹⁸ is chloro, fluoro, trifluoromethyl or methyl,

provided that R¹⁴, R¹⁵ R¹⁷ and R¹⁸ are not all fluoro when R¹³ is ethyl and R¹⁶ is H.

A preferred 5-alkyl-2-arylaminophenylacetic acid compound useful in the combinations and methods of the present invention is the compound of Formula (C67),

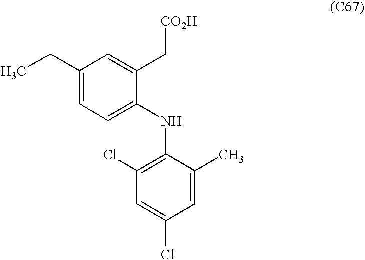

2-[(2-chloro-6-fluorophenyl)amino]-5-methyl-benzeneacetic acid, or a pharmaceutically acceptable salt thereof, or a pharmaceutically acceptable prodrug ester thereof.

Other preferred COX-2 inhibitors that can be used in the present invention have the general structure shown in formula (5),

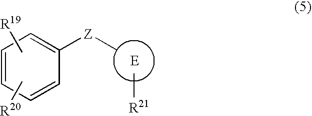

where

Z is O; E is 1-phenyl; R¹⁹ is 2-NHSO₂CH₃; R²⁰ is 4-NO₂; and there is no R²¹ group, (nimesulide), and

Z is O; E is 1-oxo-inden-5-yl; R¹⁹ is 2-F; R²⁰ is 4-F; and R²¹ is 6-NHSO₂CH₃, (flosulide); and

Z is O; E is cyclohexyl; R¹⁹ is 2-NHSO₂CH₃; R²⁰ is 5-NO₂; and there is no R²¹ group, (NS-398); and

Z is S; E is 1-oxo-inden-5-yl; R¹⁹ is 2-F; R²⁰ is 4-F; and R²¹ is 6-N⁻SO₂CH₃.Na⁺, (L-745337); and

Z is S; E is thiophen-2-yl; R¹⁹ is 4-F; there is no R²⁰ group; and R²¹ is 5-NHSO₂CH₃₁ (RWJ-63556); and

Z is O; E is 2-oxo-5(R)-methyl-5-(2,2,2-trifluoroethyl)furan-(5H)-3-yl; R¹⁹ is 3-F; R²⁰ is 4-F; and R²¹ is 4-(p-SO₂CH₃)C₆H₄, (L-784512).

Other materials that can serve as the COX-2 inhibitor of the present invention include diarylmethylidenefuran derivatives that are described in U.S. Pat. No. 6,180,651. Such diarylmethylidenefuran derivatives have the general formula shown below in formula (6):

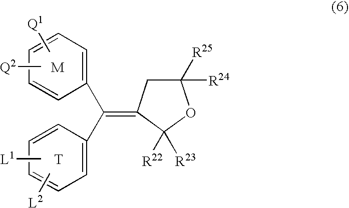

wherein:

T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;

Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,

Q¹ and Q² are methylenedioxy; or

L¹ and L² are methylenedioxy; and

R²², R²³, R²⁴, and R²⁵ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,

R²² and R²³ are 0; or,

R²⁴ and R²⁵ are 0; or,

R²², R²³, together with the carbon atom to which they are attached, form a saturated hydrocarbon ring having from 3 to 7 carbon atoms; or,

R²⁴, R²⁵, together with the carbon atom to which they are attached, form a saturated hydrocarbon ring having from 3 to 7 carbon atom;

or a pharmaceutically acceptable salt, an isomer or prodrug thereof.

Specific compounds that are useful for the COX-2 selective inhibitor include:

H1) 2-trifluoromethyl-3H-naphthopyran-3-carboxylic acid;

H2) 2-trifluoromethyl-3H-naptho[2,1-b]pyran-3-carboxylic acid;

H3) 5,7-dichloro-2-trifluoromethyl-2H-1-benzopyran-3-carboxylic acid;

H4) 6,7-dichloro-2-trifluoromethyl-2H-1-benzopyran-3-carboxylic acid;

H5) 6,8-bis(dimethylethyl)-2-trifluoromethyl-2H-1-benzopyran-3-carboxylic acid;

H6) 6,8-dibromo-2-trifluoromethyl-2H-1-benzopyran-3-carboxylic acid;

H7) 6,8-dichloro-(S)-2-trifluoromethyl-2H-1-benzopyran-3-carboxylic acid;

H8) 6,8-dichloro-2-trifluoromethyl-2H-1-benzopyran-3-carboxylic acid;

H9) 6-[(1,1-dimethylethyl)aminosulfonyl]-2-trifluoromethyl-2H-1-benzopyran-3-carboxylic acid;

H10) 6-[(2-methylpropyl)aminosulfonyl]-2-trifluoromethyl-2H-1-benzopyran-3-carboxylic acid;

H11) 6-[(4-morpholino)sulfonyl]-2-trifluoromethyl-2H-1-benzopyran-3-carboxylic acid;

H12) 6-[(dimethylamino)sulfonyl]-2-trifluoromethyl-2H-1-benzopyran-3-carboxylic acid;

H13) 6-[(methylamino)sulfonyl]-2-trifluoromethyl-2H-1-benzopyran-3-carboxylic acid;

H14) 6-[[(phenylmethyl)amino]sulfonyl]-2-trifluoromethyl-2H-1-benzopyran-3-carboxylic acid;

H15) 6-[[N-(2-furylmethyl)amino]sulfonyl]-2-trifluoromethyl-2H-1-benzopyran-3-carboxylic acid;

H16) 6-[[N-(2-phenylethyl)amino]sulfonyl]-2-trifluoromethyl-2H-1-benzopyran-3-carboxylic acid;

H17) 6-benzylsulfonyl-2-trifluoromethyl-2H-1-benzopyran-3-carboxylic acid;

H18) 6-bromo-2-trifluoromethyl-2H-1-benzopyran-3-carboxylic acid;

H19) 6-bromo-8-chloro-2-trifluoromethyl-2H-1-benzopyran-3-carboxylic acid;

H20) 6-bromo-8-methoxy-2-trifluoromethyl-2H-1-benzopyran-3-carboxylic acid;

H21) 6-chloro-2-trifluoromethyl-2H-1-benzopyran-3-carboxylic acid;

H22) 6-chloro-2-trifluoromethyl-2H-1-benzothiopyran-3-carboxylic acid;

H23) 6-chloro-7-(1,1-dimethylethyl)-2-trifluoromethyl-2H-1-benzopyran-3-carboxylic acid;

H24) 6-chloro-7-ethyl-2-trifluoromethyl-2H-1-benzopyran-3-carboxylic acid;

H25) 6-chloro-7-methyl-2-trifluoromethyl-2H-1-benzopyran-3-carboxylic acid;

H26) 6-chloro-7-phenyl-2-trifluoromethyl-2H-1-benzopyran-3-carboxylic acid;

H27) 6-chloro-8-(1-methylethyl)-2-trifluoromethyl-2H-1-benzopyran-3-carboxylic acid;

H28) 6-chloro-8-ethyl-2-trifluoromethyl-2H-1-benzopyran-3-carboxylic acid;

H29) 6-chloro-8-fluoro-2-trifluoromethyl-2H-1-benzopyran-3-carboxylic acid;

H30) 6-chloro-8-methyl-2-trifluoromethyl-2H-1-benzopyran-3-carboxylic acid;

H31) 6-iodo-2-trifluoromethyl-2H-1-benzopyran-3-carboxylic acid;

H32) 6-methylsulfonyl-2-trifluoromethyl-2H-1-benzopyran-3-carboxylic acid;

H33) 6-phenylacetyl-2-trifluoromethyl-2H-1-benzopyran-3-carboxylic acid;

H34) 6-trifluoromethoxy-2-trifluoromethyl-2H-1-benzopyran-3-carboxylic acid;

H35) 7-(1,1-dimethylethyl)-2-pentafluoroethyl-2H-1-benzopyran-3-carboxylic acid;

H36) 7-(1,1-dimethylethyl)-2-trifluoromethyl-2H-1-benzopyran-3-carboxylic acid;

H37) 7-(1-methylethyl)-2-trifluoromethyl-2H-1-benzopyran-3-carboxylic acid;

H38) 7,8-dimethyl-2-trifluoromethyl-2H-1-benzopyran-3-carboxylic acid;

H39) 7-phenyl-2-trifluoromethyl-2H-1-benzopyran-3-carboxylic acid;

H40) 8-(1-methylethyl)-2-trifluoromethyl-2H-1-benzopyran-3-carboxylic acid;

H41) 8-bromo-5-fluoro-2-trifluoromethyl-2H-1-benzopyran-3-carboxylic acid;

H42) 8-bromo-6-fluoro-2-trifluoromethyl-2H-1-benzopyran-3-carboxylic acid;

H43) 8-bromo-6-methyl-2-trifluoromethyl-2H-1-benzopyran-3-carboxylic acid;

H44) 8-chloro-2-trifluoromethyl-2H-1-benzopyran-3-carboxylic acid;

H45) 8-chloro-5,6-dimethyl-2-trifluoromethyl-2H-1-benzopyran-3-carboxylic acid;

H46) 8-chloro-6-[[(phenylmethyl)amino]sulfonyl]-2-trifluoromethyl-2H-1-benzopyran-3-carboxylic acid;

H47) 8-chloro-6-methoxy-2-trifluoromethyl-2H-1-benzopyran-3-carboxylic acid;

H48) 8-chloro-6-methyl-2-trifluoromethyl-2H-1-benzopyran-3-carboxylic acid;

H49) 8-phenyl-2-trifluoromethyl-2H-1-benzopyran-3-carboxylic acid;

or a pharmaceutically acceptable salt of the compound.

COX-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), S-2474 (Shionogi), DFP (Merck), E-6087 (Laboratorias Dr Esteve SA), GW-406381 (Glaxo Welcome), LAS-33815 (Almirall Prodesfarma), and SVT-2016 (Laboratorios Salvat SA).

Various classes of COX-2 inhibitors useful in the present invention can be prepared as follows. Pyrazoles can be prepared by methods described in WO 95/15316. Pyrazoles can further be prepared by methods described in WO 95/15315. Pyrazoles can also be prepared by methods described in WO 96/03385.

Thiophene analogs useful in the present invention can be prepared by methods described in WO 95/00501. Preparation of thiophene analogs is also described in WO 94/15932.

Oxazoles useful in the present invention can be prepared by the methods described in WO 95/00501. Preparation of oxazoles is also described in WO 94/27980.

Isoxazoles useful in the present invention can be prepared by the methods described in WO 96/25405.

Imidazoles useful in the present invention can be prepared by the methods described in WO 96/03388. Preparation of imidazoles is also described in WO 96/03387.

Cyclopentene COX-2 inhibitors useful in the present invention can be prepared by the methods described in U.S. Pat. No. 5,344,991. Preparation of cyclopentene COX-2 inhibitors is also described in WO 95/00501.

Terphenyl compounds useful in the present invention can be prepared by the methods described in WO 96/16934.

Thiazole compounds useful in the present invention can be prepared by the methods described in WO 96/03,392.

Pyridine compounds useful in the present invention can be prepared by the methods described in WO 96/03392. Preparation of pyridine compounds is also described in WO 96/24,585.

Benzopyranopyrazolyl compounds useful in the present invention can be prepared by the methods described in WO 96/09304.

Chromene compounds useful in the present invention can be prepared by the methods described in WO 98/47890. Preparation of chromene compounds is also described in WO 00/23433. Chromene compounds can further be prepared by the methods described in U.S. Pat. No. 6,077,850. Preparation of chromene compounds is further described in U.S. Pat. No. 6,034,256.

Arylpyridazinones useful in the present invention can be prepared by the methods described in WO 00/24719. Preparation of arylpyridazinones is also described in WO 99/10332. Arylpyridazinones can further be prepared by the methods described in WO 99/10331.

5-Alkyl-2-arylaminophenylacetic acids and derivatives useful in the present invention can be prepared by the methods described in WO 99/11605.

Diarylmethylidenefuran derivative COX-2 selective inhibitors useful in the present invention can be prepared by the methods described in U.S. Pat. No. 6,180,651.

The celecoxib used in the compositions and methods of the present invention can be prepared in the manner set forth in U.S. Pat. No. 5,466,823.

The valdecoxib used in the compositions and methods of the present invention can be prepared in the manner set forth in U.S. Pat. No. 5,633,272.

The parecoxib used in the compositions and methods of the present invention can be prepared in the manner set forth in U.S. Pat. No. 5,932,598.

The rofecoxib used in the compositions and methods of the present invention can be prepared in the manner set forth in U.S. Pat. No. 5,474,995.

The deracoxib used in the compositions and methods of the present invention can be prepared in the manner set forth in U.S. Pat. No. 5,521,207.

The etoricoxib used in the compositions and methods of the present invention can be prepared in the manner set forth in WO 98/03484.

The meloxicam used in the compositions and methods of the present invention can be prepared in the manner set forth in U.S. Pat. No. 4,233,299.

The compound 4-(4-cyclohexyl-2-methyloxazol-5-yl)-2-fluorobenzenesulfonamide used in the compositions and methods of the present invention can be prepared in the manner set forth in U.S. Pat. No. 5,994,381.

The compound 2-(3,4-difluorophenyl)-4-(3-hydroxy-3-methylbutoxy)-5-[4-(methylsulfonyl)phenyl]-3(2H)-pyridazinone used in the compositions and methods of the present invention can be prepared in the manner set forth in WO 00/24719.

The compound 2-(3,5-difluorophenyl)-3-[4-(methylsulfonyl)phenyl]-2-cyclopenten-1-one used in the compositions and methods of the present invention can be prepared in the manner set forth in EP 863134.

The compound 2-[(2-chloro-6-fluorophenyl)amino]-5-methyl-benzeneacetic acid used in the compositions and methods of the present invention can be prepared in the manner set forth in WO 99/11605.

The compound N-[2-(cyclohexyloxy)-4-nitrophenyl]methanesulfonamide used in the compositions and methods of the present invention can be prepared in the manner set forth in U.S. Pat. No. 4,885,367.

The compound (3Z)-3-[(4-chlorophenyl)[4-(methylsulfonyl)phenyl]methylene]dihydro-2(3H)-furanone used in the compositions and methods of the present invention can be prepared in the manner set forth in U.S. Pat. No. 6,180,651.

The above individual references are each herein individually incorporated by reference.

The expression “N-methyl-D-aspartate receptor” is to be understood as including all of the binding site subcategories associated with the NMDA receptor, e.g., the glycine-binding site, the phenylcyclidine (PCP)-binding site, the polyamine associated site, the polyamine site, the glutamate-binding site, the sigma site, the NR2B receptor site, etc., as well as the NMDA ion channel. Thus, the invention herein contemplates the use of certain selected substances that block a NMDA receptor binding site, e.g., dextromethorphan, or that block the NMDA ion channel.

The structures of selected NMDA antagonists are listed in Table No. 2 below.

TABLE NO. 2
Selected NMDA antagonists

| Compound | |
|---|---|
| Number | Structure |
| N1 | 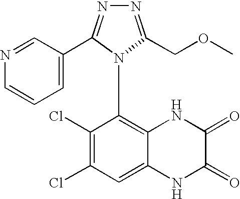 |
| N2 | 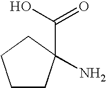 |
| N3 | 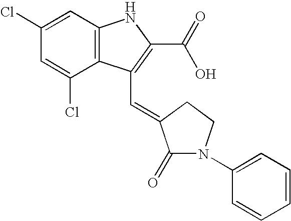 |
| N4 | 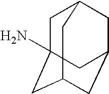 |
| N5 | 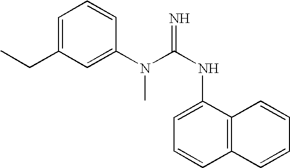 |
| N6 | 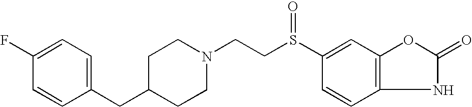 |
| N7 | 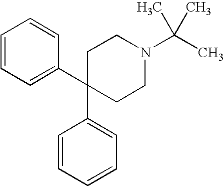 |
| N8 | 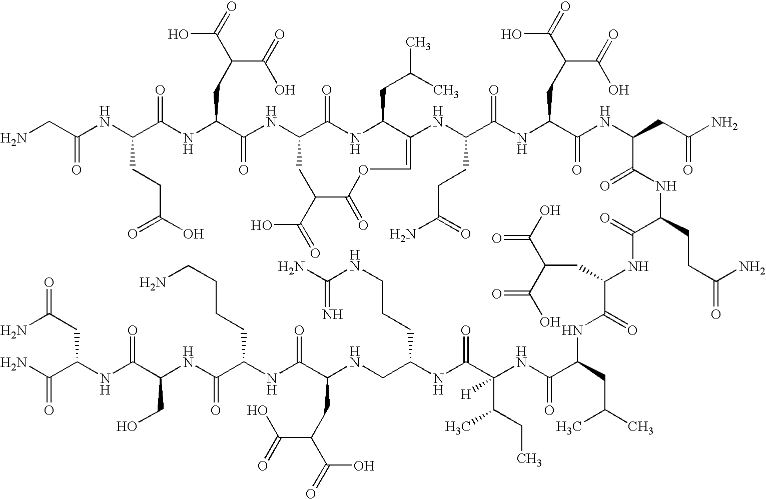 |
| N9 | 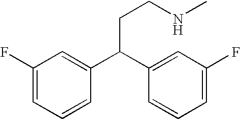 |
| N10 | 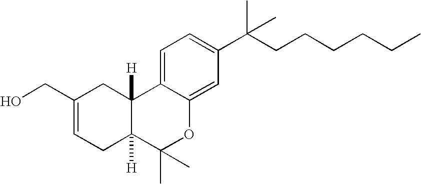 |
| N11 | 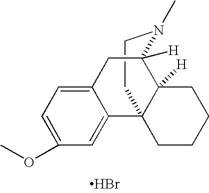 |
| N12 | 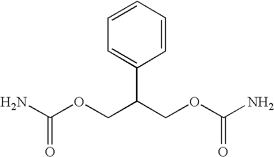 |
| N13 | 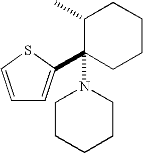 |
| N16 | 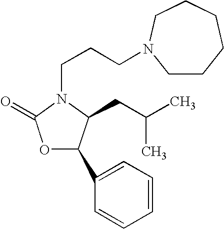 |
| N17 | 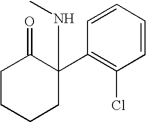 |
| N18 | 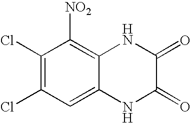 |
| N19 | 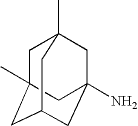 |
| N20 | 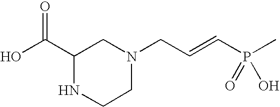 |
| N21 | 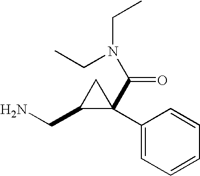 |
| N22 | 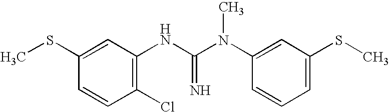 |
| N23 | 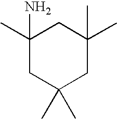 |
| N24 | 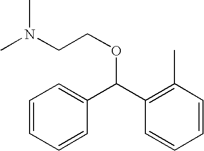 |
| N25 | 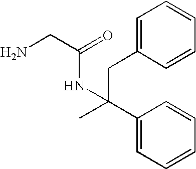 |
| N26 | 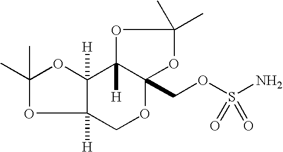 |
| N28 | 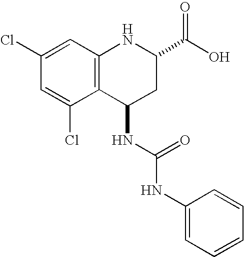 |
| N29 | 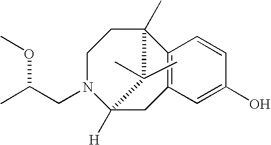 |
| N30 | 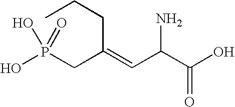 |
| N31 | 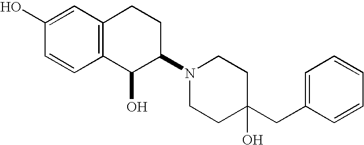 |
| N32 | 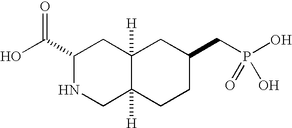 |
| N33 | 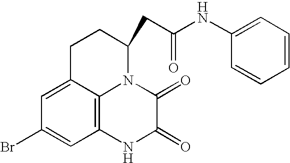 |
| N34 | 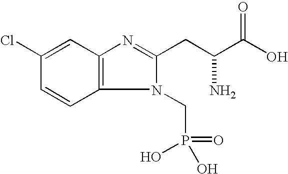 |
| N35 | 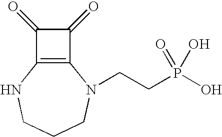 |
| N36 | 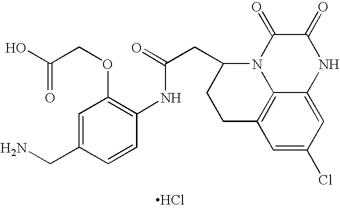 |
| N37 | 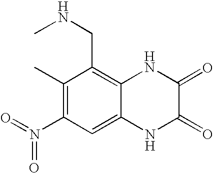 |
| N38 | 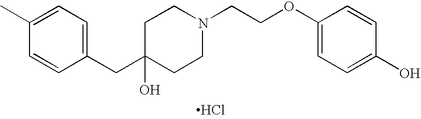 |
| N39 | 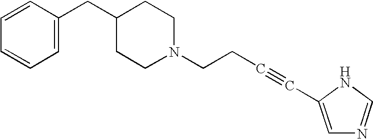 |
| N40 | 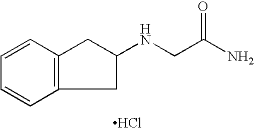 |
| N41 | 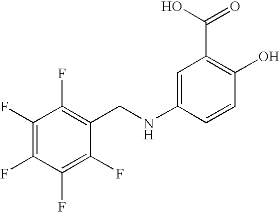 |
| N42 | 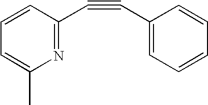 |
| N43 | 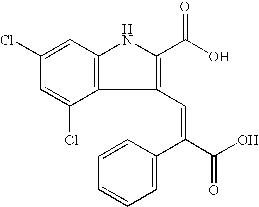 |
| N44 | 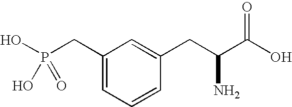 |
| N45 | 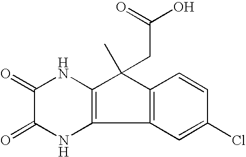 |
| N46 | 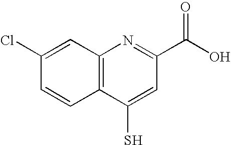 |
| N47 | 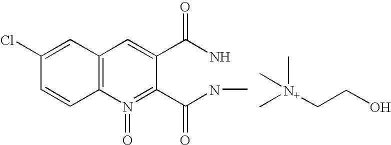 |
| N52 | 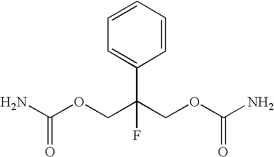 |
| N54 | 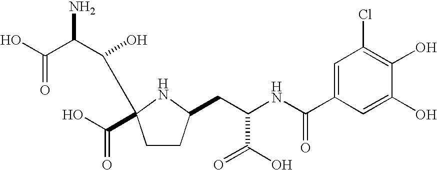 |
| N55 | 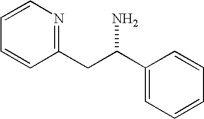 |
| N56 | 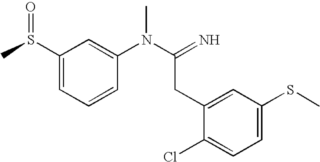 |
| N58 | 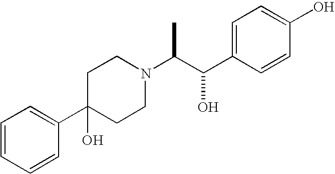 |
| N59 | 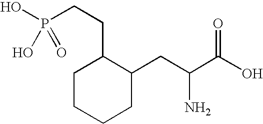 |
| N60 | 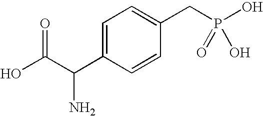 |

The names, CAS registry numbers and references for selected NMDA antagonists are listed in Table No. 3 below. The individual references in Table No. 3 are each herein individually incorporated by reference.

TABLE No. 3
Selected NMDA antagonist Names, CAS
Registry Numbers and References

| | | CAS | |
|---|---|---|---|
| Compound | | Registry | |
| Number | Name(s) | Number | Reference |
| N1 | (-)-6,7-dichloro- | 197093-13-3 | US 6333326 |
| | 1,4-dihydro-5-[3- | | |
| | (methoxymethyl)-5- | | |
| | (3-pyridinyl)-4H- | | |
| | 1,2,4-triazol-4- | | |
| | yl]-2,3- | | |
| | quinoxalinedione | | |
| | (UK-315716) | | |
| | (Pfizer) | | |
| N2 | 1- | 52-52-8 | Zelinsky, |
| | aminocyclopentane- | | Stadnikoff, |
| | carboxylic acid | | Z. |
| | (ACPC) | | Physiol. |
| | | | Chem. 75, |
| | | | 350 (1911) |
| N3 | 4,6-dichloro-3- | 166974-22-7 | WO 9510517 |
| | [(E)-(2-oxo-1- | | |
| | phenyl-3- | | |
| | pyrrolidinylidene)- | | |
| | methyl]-1H-indole-2- | | |
| | carboxylic acid (GV | | |
| | 196771) (Glaxo | | |
| | Wellcome) | | |
| N4 | amantadine | 768-94-5 | H. Stetter |
| | | | et al., |
| | | | Ber. 93, |
| | | | 226 (1960) |
| N5 | aptiganel | 137159-92-3 | WO 9112797 |
| N6 | besonprodil | 253450-09-8 | US 6284774 |
| | (PD-196860) | | |
| | (CI1041) | | |
| N7 | budipine | 57982-78-2 | US 4016280 |
| N8 | conantokin | 93438-65-4 | WO 9803541 |
| | G(Cognetix) | | |
| N9 | delucemine (NPS | 186495-49-8 | US 6071970 |
| | Pharmaceuticals) | | |
| N10 | dexanabinol | 112924-45-5 | US 4876276 |
| | (HU-211) | | |
| N11 | dextromethorphan | 125-71-3 | US 2676177 |
| N12 | felbamate | 25451-15-4 | US 4868327 |
| N13 | gacyclidine | 68134-81-6 | US 5179109 |
| | (Beaufour-Ipsen) | | |
| N14 | glycine (AZD- | 56-40-6 | |
| | 4282)(Astra Zeneca) | | |
| N15 | GW-468816 (Glaxo | | |
| | SmithKline) | | |
| N16 | ipenoxazone (Nippon | 104454-71-9 | JP 2649947 |
| | Chemiphar) | | |
| N17 | ketamine | 6740-88-1 | US 3254124 |
| N18 | licostinel | 153504-81-5 | US 5622952 |
| N19 | memantine | 19982-08-2 | US 3391142 |
| N20 | midafotel | 117414-74-1 | GB 2201676 |
| N21 | milnacipran | 92623-85-3 | EP 200638 |
| N22 | N′-[2-chloro-5- | 160754-76-7 | WO |
| | (methylthio)phenyl]-N- | | 9427591 |
| | methyl-N-[3- | | |
| | (methylthio)phenyl]- | | |
| | guanidine(CNS- | | |
| | 5161) (CeNeS | | |
| | Pharmaceuticals) | | |
| N23 | neramexane (Merz) | 219810-59-0 | US |
| | | | 6034134 |
| N24 | orphenadrine | 83-98-7 | Harms, |
| | | | Nauta, J. |
| | | | Med. |
| | | | Pharm. |
| | | | Chem. 2, |
| | | | 57 (1960) |
| N25 | remacemide | 128298-28-2 | US 5331007 |
| N26 | topiramate | 97240-79-4 | US |
| | | | 4513006 |
| N27 | YKP 509 (SK Corp) | | |
| N28 | (2R,4S)-rel-5,7- | 139051-78-8 | US 5231102 |
| | dichloro-1,2,3,4- | | |
| | tetrahydro-4- | | |
| | [[(phenylamino)- | | |
| | carbonyl]amino]-2- | | |
| | quinolinecarboxylic | | |
| | acid (L 689560) | | |
| N29 | (2R,6S)- | 193278-48-7 | Grauert, |
| | 1,2,3,4,5,6- | | M., et al. |
| | hexahydro-3-[(2S)- | | J. Med. |
| | 2-methoxypropyl]- | | Chem. |
| | 6,11,11-trimethyl- | | (1997), |
| | 2,6-methano-3- | | 40(18), |
| | benzazocin-9-ol | | 2922-2930 |
| | (BI-II-277-CL) | | |
| N30 | (3E)-2-amino-4- | 132472-31-2 | EP 233154 |
| | (phosphonomethyl)- | | |
| | 3-heptenoic acid | | |
| | (CGP-39653) | | |
| N31 | (3R,4S)-rel-3,4- | 138047-56-0 | WO 9112005 |
| | dihydro-3-[4- | | |
| | hydroxy-4- | | |
| | (phenylmethyl)-1- | | |
| | piperidinyl]-2H-1- | | |
| | benzopyran-4,7-diol | | |
| | (CP-283097) | | |
| N32 | (3S,4aR,6S,8aR)- | 137433-06-8 | US 5461156 |
| | decahydro-6- | | |
| | (phosphonomethyl)- | | |
| | 3- | | |
| | isoquinolinecarbox- | | |
| | ylic acid (LY- | | |
| | 235959) | | |
| N33 | (R)-9-bromo- | 158328-22-4 | US 5616586 |
| | 2,3,6,7-tetrahydro- | | |
| | 2,3-dioxo-N-phenyl- | | |
| | 1H,5H-pyrido[1,2,3- | | |
| | de]quinoxaline-5- | | |
| | acetamide (SM | | |
| | 31900) | | |
| N34 | (αR)-α-amino-5- | 143850-75-3 | US 5124319 |
| | chloro-1- | | |
| | (phosphonomethyl)- | | |
| | 1H-benzimidazole-2- | | |
| | propanoic acid | | |
| | (EAB-318) | | |
| N35 | [2-(8,9-dioxo-2,6- | 144912-63-0 | EP 496561 |
| | diazabicyclo[5.2.0] | | |
| | non-1(7)-en-2- | | |
| | yl)ethyl]- | | |
| | phosphonic acid | | |
| | (EAA-090) | | |
| N36 | [5-(aminomethyl)-2- | 161292-39-3 | US 5719152 |
| | [[[(5S)-9-chloro- | | |
| | 2,3,6,7-tetrahydro- | | |
| | 2,3-dioxo-1H,5H- | | |
| | pyrido[1,2,3- | | |
| | de]quinoxalin-5- | | |
| | yl]acetyl]amino]- | | |
| | phenoxy]-acetic acid, | | |
| | monohydrochloride | | |
| | (SM-18400) | | |
| | (Sumitomo) | | |
| N37 | 1,4-dihydro-6- | 200430-63-3 | WO 9746539 |
| | methyl-5- | | |
| | [(methylamino)- | | |
| | methyl]- | | |
| | 7-nitro-2,3- | | |
| | quinoxalinedione | | |
| | (PD 165650) | | |
| | (Pfizer) | | |
| N38 | l-[2-(4- | 193356-17-1 | US 6124323 |
| | hydroxyphenoxy)- | | |
| | ethyl]-4-[(4- | | |
| | methylphenyl)- | | |
| | methyl]-4- | | |
| | piperidinol, | | |
| | hydrochloride (CO | | |
| | 101244) | | |
| N39 | 1-[4-(1H-imidazol- | 252374-41-7 | Wright, J. |
| | 4-yl)-3-butynyl]-4- | | L., et al. |
| | (phenylmethyl)- | | Bioorganic |
| | piperidine (PD | | & |
| | 188669) | | Medicinal |
| | | | Chemistry |
| | | | Letters |
| | | | (1999), |
| | | | 9(19), |
| | | | 2815-2818. |
| N40 | 2-[(2,3-dihydro-1H- | 202914-18-9 | US 6114391 |
| | inden-2-yl)amino]- | | |
| | acetamide, | | |
| | monohydrochloride | | |
| | (CHF-3381) | | |
| N41 | 2-hydroxy-5- | 369640-27-7 | WO 0179153 |
| | [[(pentafluoro- | | |
| | phenyl)methyl]- | | |
| | amino]- | | |
| | benzoic acid (PBAS) | | |
| N42 | 2-methyl-6- | 96206-92-7 | Nishiwaki, |
| | (phenylethynyl)- | | N., et al. |
| | pyridine(MPEP) | | Chem. |
| | | | Lett. |
| | | | (1989), |
| | | | (5), 773- |
| | | | 6. |
| N43 | 3- | 142235-88-9 | Mueller, |
| | (phosphonomethyl)- | | W., et al. |
| | L-phenylalanine (PD | | Helv. |
| | 130527) | | Chim. Acta |
| | | | (1992), |
| | | | 75(3), |
| | | | 855-64. |
| N44 | 3-[(1E)-2-carboxy- | 161230-88-2 | WO 9427964 |
| | 2-phenylethenyl]- | | |
| | 4,6-dichloro-1H- | | |
| | indole-2-carboxylic | | |
| | acid (MDL 105519) | | |
| N45 | 6-chloro-2,3,4,9- | 173186-99-7 | US 5922716 |
| | tetrahydro-9- | | |
| | methyl-2,3-dioxo- | | |
| | 1H-indeno[1,2- | | |
| | b]pyrazine-9-acetic | | |
| | acid (RPR 118723) | | |
| N46 | 7-chlorothiokynurenic | 135025-56-8 | US 5250541 |
| | acid | | |
| N47 | 8-chloro-2,3- | 202807-80-5 | US 5776935 |
| | dihydropyridazino- | | |
| | [4,5-b]quinoline- | | |
| | 1,4-dione 5-oxide | | |
| | salt with 2-hydroxy- | | |
| | N,N,N-trimethyl- | | |
| | ethanaminium | | |
| | (1:1)(MRZ 2/576) | | |
| N48 | ACEA-1286 (Pfizer) | | |
| N49 | AY 12316 (Wyeth | | |
| | Ayerst) | | |
| N50 | DD-20207 | | |
| | (DiverDrugs) | | |
| N51 | DD-B4 (DiverDrugs) | | |
| N52 | fluorofelbamate | 726-99-8 | WO |
| | | | 200047202 |
| N53 | GV 117164X (Glaxo | | |
| | Wellcome) | | |
| N54 | kaitocephalin | 198710-92-8 | US 6171829 |
| N55 | lanicemine | 153322-05-5 | US 5455259 |
| N56 | N′-[2-chloro-5- | 342047-49-8 | Padmanabha |
| | (methylthio)- | | n, S., et |
| | phenyl]-N-methyl- | | al. |
| | N-[3-[(R)- | | Tetrahedro |
| | methylsulfinyl]- | | n: |
| | phenyl]-guanidine | | Asymmetry |
| | (CNS 5788) | | (2000), |
| | | | 11(17), |
| | | | 3455-3457. |
| N57 | NC-1210 (Queens | | |
| | University at | | |
| | Kingston) | | |
| N58 | traxoprodil (CP- | 134234-12-1 | EP 398578 |
| | 101606) | | |
| N59 | α-amino-2-(2- | 117571-54-7 | US 4761405 |
| | phosphonoethyl)- | | |
| | cyclohexane- | | |
| | propanoic acid | | |
| | (NPC-12626) | | |
| N60 | α-amino-4- | 120667-19-8 | US 5175153 |
| | (phosphonomethyl)- | | |
| | benzeneacetic acid | | |
| | (PD 129653) | | |

Preferred NMDA antagonists for the present invention include (−)-6,7-dichloro-1,4-dihydro-5-[3-(methoxymethyl)-5-(3-pyridinyl)-4H-1,2,4-triazol-4-yl]-2,3-quinoxalinedione (UK-315716); 1-aminocyclopentane-carboxylic acid (ACPC); 4,6-dichloro-3-[(E)-(2-oxo-1-phenyl-3-pyrrolidinylidene)methyl]-1H-indole-2-carboxylic acid (GV 196771); amantadine; aptiganel; besonprodil; budipine; conantokin G; delucemine; dexanabinol (HU-211); dextromethorphan; felbamate; gacyclidine; glycine (AZD-4282); GW-468816; ipenoxazone; ketamine; licostinel; memantine; midafotel; milnacipran; N′-[2-chloro-5-(methylthio)phenyl]-N-methyl-N-[3-(methylthio)phenyl]-guanidine (CNS-5161); neramexane; orphenadrine; PD-196860; remacemide; topiramate; and YKP 509.

Especially preferred NMDA antagonists for the present invention include amantadine; budipine; dextromethorphan; felbamate; ketamine; memantine; milnacipran; orphenadrine; and topiramate.

The compounds useful in the present invention can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

Also included in the methods, combinations and compositions of the present invention are the isomeric forms and tautomers of the described compounds and the pharmaceutically-acceptable salts thereof. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, b-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

Also included in the methods, combinations and compositions of the present invention are the prodrugs of the described compounds and the pharmaceutically-acceptable salts thereof. The term “prodrug” refers to drug precursor compounds which, following administration to a subject and subsequent absorption, are converted to an active species in vivo via some process, such as a metabolic process. Other products from the conversion process are easily disposed of by the body. More preferred prodrugs produce products from the conversion process that are generally accepted as safe. A nonlimiting example of a “prodrug” that will be useful in the methods, combinations and compositions of the present invention is parecoxib (N-[[4-(5-methyl-3-phenyl-4-isoxazolyl)phenyl]sulfonyl]propanamide).

The methods and combinations of the present invention are useful for the treatment, prevention or inhibition of neuropathic pain.

A “therapeutically effective amount” is intended to qualify the amount of a COX-2 inhibiting agent and a NMDA antagonist required to treat, prevent or inhibit neuropathic pain or relieve to some extent one or more of the symptoms of neuropathic pain, including, but not limited to: 1) hypersensitivity at the site of injury; 2) mechanoallodynia; 3) thermal hyperalgesia; 4) hyperpathia; 5) extraterritoriality (regional distribution of pain) in the case of complex regional pain syndrome/reflex sympathetic dystrophy; and 6) associated neurogenic inflammation, autonomic dysregulation, and motor phenomena that are especially found in complex regional pain syndrome/reflex sympathetic dystrophy.

Neuropathic pain or nociceptive central pain may be caused by direct injury to the brain or spinal cord, as well as by damage to peripheral nociceptive nerve endings in soft tissues, plexuses, or the nerves themselves. Neuropathic pain may follow stroke, spinal cord injury, and the progress of multiple sclerosis, brain injury or trauma to the central nervous system.

The term “treatment,” in relation to neuropathic pain is defined as the administration of a combination of the present invention to alleviate the symptoms of the condition.

The term “prevention,” in relation to neuropathic pain, implies the administration of a combination of the present invention to prevent the development of neuropathic pain through central sensitization. This prevention may take the form of preemptive analgesia for postoperative pain relief or the prevention of the development of central sensitization from ongoing peripheral nociceptive pain.

The term “inhibition,” in the context of neuropathic pain may be assessed by the reduction in the perceived severity of the sensation of central pain in the subject.

The term “central sensitization” refers to persistent post injury changes in the central nervous system that result in pain hypersensitivity.

The phrases “low dose” or “low dose amount”, in characterizing a therapeutically effective amount of the COX-2 selective inhibitor and the NMDA antagonist or therapy in the combination therapy, defines a quantity of such agent, or a range of quantity of such agent, that is capable of reducing the discomfort of neuropathic pain while optionally reducing or avoiding one or more side effects of monotherapy with a NMDA antagonist or other pain-relieving agent. Side effects of NMDA antagonists that the selected combinations of the present invention may reduce or avoid are motor deficits, sedation, psychomimetic effects, addiction and impairment of learning and memory in cognitive tasks.

The phrase “adjunctive therapy” encompasses treatment of a subject with agents that reduce or avoid side effects associated with the combination therapy of the present invention.

Dosages, Formulations and Routes of Administration

### Dosages

Dosage levels of the source of a COX-2 inhibiting agent (e.g., a COX-2 selective inhibiting agent or a prodrug of a COX-2 selective inhibiting agent) on the order of about 0.1 mg to about 10,000 mg of the active ingredient compound are useful in the treatment of the above conditions, with preferred levels of about 1.0 mg to about 1,000 mg. While the dosage of active compound administered to a warm-blooded animal (a mammal), is dependent on the species of that mammal, the body weight, age, and individual condition, and on the route of administration, the unit dosage for oral administration to a mammal of about 50 to 70 kg may contain between about 5 and 500 mg of the active ingredient (for example, COX-189). The amount of active ingredient that may be combined with a NMDA antagonist to produce a single dosage form will vary depending upon the host treated and the particular mode of administration.

A total daily dose of a NMDA antagonist can generally be in the range of from about 0.001 to about 10,000 mg/day in single or divided doses. It is understood, however, that specific dose levels of the therapeutic agents or therapeutic approaches of the present invention for any particular patient depends upon a variety of factors including the activity of the specific compound employed, the age, body weight, general health, sex, and diet of the patient, the time of administration, the rate of excretion, the drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of neuropathic pain in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 μM, one will desire to administer an amount of the drug that is effective to provide about a 10 μM concentration in vivo. Determination of these parameters is well within the skill of the art.

### Formulations and Routes of Administration

Effective formulations and administration procedures are well known in the art and are described in standard textbooks.

The COX-2 inhibiting agents or the NMDA antagonists can be formulated as a single pharmaceutical composition or as independent multiple pharmaceutical compositions. Pharmaceutical compositions according to the present invention include those suitable for oral, inhalation spray, rectal, topical, buccal (e.g., sublingual), or parenteral (e.g., subcutaneous, intramuscular, intravenous, intrathecal, intramedullary and intradermal injections, or infusion techniques) administration, although the most suitable route in any given case will depend on the nature and severity of the condition being treated and on the nature of the particular compound which is being used. In most cases, the preferred route of administration is oral or parenteral.

Compounds and composition of the present invention can then be administered orally, by inhalation spray, rectally, topically, buccally or parenterally in dosage unit formulations containing conventional nontoxic pharmaceutically acceptable carriers, adjuvants, and vehicles as desired. The compounds of the present invention can be administered by any conventional means available for use in conjunction with pharmaceuticals, either as individual therapeutic compounds or as a combination of therapeutic compounds.

Pharmaceutically acceptable salts are particularly suitable for medical applications because of their greater aqueous solubility relative to the parent compound. Such salts must clearly have a pharmaceutically acceptable anion or cation.

The compounds useful in the methods, combinations and compositions of the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well-known techniques of pharmacy, consisting essentially of admixing the components.

The amount of compound in combination that is required to achieve the desired biological effect will, of course, depend on a number of factors such as the specific compound chosen, the use for which it is intended, the mode of administration, and the clinical condition of the recipient.

The compounds of the present invention can be delivered orally either in a solid, in a semi-solid, or in a liquid form. Dosing for oral administration may be with a regimen calling for single daily dose, or for a single dose every other day, or for multiple, spaced doses throughout the day. For oral administration, the pharmaceutical composition may be in the form of, for example, a tablet, capsule, suspension, or liquid. Capsules, tablets, etc., can be prepared by conventional methods well known in the art. The pharmaceutical composition is preferably made in the form of a dosage unit containing a particular amount of the active ingredient or ingredients. Examples of dosage units are tablets or capsules, and may contain one or more therapeutic compounds in an amount described herein. For example, in the case of a NMDA antagonist, the dose range may be from about 0.01 mg to about 5,000 mg or any other dose, dependent upon the specific inhibitor, as is known in the art. When in a liquid or in a semi-solid form, the combinations of the present invention can, for example, be in the form of a liquid, syrup, or contained in a gel capsule (e.g., a gel cap). In one embodiment, when a NMDA antagonist is used in a combination of the present invention, the NMDA antagonist can be provided in the form of a liquid, syrup, or contained in a gel capsule. In another embodiment, when a COX-2 inhibiting agent is used in a combination of the present invention, the COX-2 inhibiting agent can be provided in the form of a liquid, syrup, or contained in a gel capsule.

Oral delivery of the combinations of the present invention can include formulations, as are well known in the art, to provide prolonged or sustained delivery of the drug to the gastrointestinal tract by any number of mechanisms. These include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. For some of the therapeutic compounds useful in the methods, combinations and compositions of the present invention the intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethylcellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one therapeutic compound useful in the present invention; as a powder or granules; as a solution or a suspension in an aqueous or non-aqueous liquid; or as an oil-in-water or water-in-oil emulsion. As indicated, such compositions can be prepared by any suitable method of pharmacy, which includes the step of bringing into association, the active compound(s) and the carrier (which can constitute one or more accessory ingredients). In general, the compositions are prepared by uniformly and intimately admixing the active compound with a liquid or finely divided solid carrier, or both, and then, if necessary, shaping the product. For example, a tablet can be prepared by compressing or molding a powder or granules of the compound, optionally with one or more assessory ingredients. Compressed tablets can be prepared by compressing, in a suitable machine, the compound in a free-flowing form, such as a powder or granules optionally mixed with a binder, lubricant, inert diluent and/or surface active/dispersing agent(s). Molded tablets can be made by molding, in a suitable machine, the powdered compound moistened with an inert liquid diluent.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sublingual) administration include lozenges comprising a compound of the present invention in a flavored base, usually sucrose, and acacia or tragacanth, and pastilles comprising the compound in an inert base such as gelatin and glycerin or sucrose and acacia.

Pharmaceutical compositions suitable for parenteral administration conveniently comprise sterile aqueous preparations of a compound of the present invention. These preparations are preferably administered intravenously, although administration can also be effected by means of subcutaneous, intramuscular, or intradermal injection or by infusion. Such preparations can conveniently be prepared by admixing the compound with water and rendering the resulting solution sterile and isotonic with the blood. Injectable compositions according to the invention will generally contain from 0.1 to 10% w/w of a compound disclosed herein.

Injectable preparations, for example, sterile injectable aqueous or oleaginous suspensions may be formulated according to the known art using suitable dispersing or setting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active ingredients may also be administered by injection as a composition wherein, for example, saline, dextrose, or water may be used as a suitable carrier. A suitable daily dose of each active therapeutic compound is one that achieves the same blood serum level as produced by oral administration as described above.

The dose of any of these therapeutic compounds can be conveniently administered as an infusion of from about 10 ng/kg body weight to about 10,000 ng/kg body weight per minute. Infusion fluids suitable for this purpose can contain, for example, from about 0.1 ng to about 10 mg, preferably from about 1 ng to about 10 mg per milliliter. Unit doses can contain, for example, from about 1 mg to about 10 g of the compound of the present invention. Thus, ampoules for injection can contain, for example, from about 1 mg to about 100 mg.

Pharmaceutical compositions suitable for rectal administration are preferably presented as unit-dose suppositories. These can be prepared by admixing a compound or compounds of the present invention with one or more conventional solid carriers, for example, cocoa butter, synthetic mono- di- or triglycerides, fatty acids and polyethylene glycols that are solid at ordinary temperatures but liquid at the rectal temperature and will therefore melt in the rectum and release the drug; and then shaping the resulting mixture.

Pharmaceutical compositions suitable for topical application to the skin preferably take the form of an ointment, cream, lotion, paste, gel, spray, aerosol, or oil. Carriers which can be used include petroleum jelly (e.g., Vaseline), lanolin, polyethylene glycols, alcohols, and combinations of two or more thereof. The active compound or compounds are generally present at a concentration of from 0.1 to 50% w/w of the composition, for example, from 0.5 to 2%.

Transdermal administration is also possible. Pharmaceutical compositions suitable for transdermal administration can be presented as discrete patches adapted to remain in intimate contact with the epidermis of the recipient for a prolonged period of time. Such patches suitably contain a compound or compounds of the present invention in an optionally buffered, aqueous solution, dissolved and/or dispersed in an adhesive, or dispersed in a polymer. A suitable concentration of the active compound or compounds is about 1% to 35%, preferably about 3% to 15%. As one particular possibility, the compound or compounds can be delivered from the patch by electrotransport or iontophoresis, for example, as described in Pharmaceutical Research, 3(6), 318 (1986).

In any case, the amount of active ingredients that can be combined with carrier materials to produce a single dosage form to be administered will vary depending upon the host treated and the particular mode of administration.

In combination therapy, administration of two or more of the therapeutic agents useful in the methods, combinations and compositions of the present invention may take place sequentially in separate formulations, or may be accomplished by simultaneous administration in a single formulation or in a separate formulation. Independent administration of each therapeutic agent may be accomplished by, for example, oral, inhalation spray, rectal, topical, buccal (e.g., sublingual), or parenteral (e.g., subcutaneous, intramuscular, intravenous, intrathecal, intramedullary and intradermal injections, or infusion techniques) administration. The formulation may be in the form of a bolus, or in the form of aqueous or non-aqueous isotonic sterile injection solutions or suspensions. Solutions and suspensions may be prepared from sterile powders or granules having one or more pharmaceutically-acceptable carriers or diluents, or a binder such as gelatin or hydroxypropylmethyl cellulose, together with one or more of a lubricant, preservative, surface active or dispersing agent. The therapeutic compounds may further be administered by any combination of, for example, oral/oral, oral/parenteral, or parenteral/parenteral route.

The therapeutic compounds which make up the combination therapy may be a combined dosage form or in separate dosage forms intended for substantially simultaneous oral administration. The therapeutic compounds, which make up the combination therapy may also be administered sequentially, with either therapeutic compound being administered by a regimen calling for two step ingestion. Thus, a regimen may call for sequential administration of the therapeutic compounds with spaced-apart ingestion of the separate, active agents. The time period between the multiple ingestion steps may range from, for example, a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the patient. Circadian variation of the target molecule concentration may also determine the optimal dose interval. The therapeutic compounds of the combined therapy whether administered simultaneously, substantially simultaneously, or sequentially, may involve a regimen calling for administration of one therapeutic compound by oral route and another therapeutic compound by intravenous route. Whether the therapeutic compounds of the combined therapy are administered orally, by inhalation spray, rectally, topically, buccally (e.g., sublingual), or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components. Examples of suitable pharmaceutically-acceptable formulations containing the therapeutic compounds are given above., Additionally, drug formulations are discussed in, for example. Hoover, John E., Remington's Pharmaceutical Sciences, Mack Publishing Co., Easton, Pa. 1975. Another discussion of drug formulations can be found in Libermann, H. A. and Lachman, L., Eds., Pharmaceutical Dosage Forms, Marcel Decker, New York, N.Y., 1980.

### Illustration 10

Table 4 illustrates examples of some combinations of the present invention wherein the combination comprises an amount of a COX-2 selective inhibitor source and an amount of a NMDA antagonist wherein the amounts together comprise a therapeutically effective amount of the compounds.

TABLE No. 4
Combinations of COX-2 selective inhibiting
agents and NMDA antagonists.

| Example | COX-2 | NMDA |
|---|---|---|
| Number | Inhibitor | Antagonist |
| 1 | C1 | N1 |
| 2 | C1 | N2 |
| 3 | C1 | N3 |
| 4 | C1 | N4 |
| 5 | C1 | N5 |
| 6 | C1 | N6 |
| 7 | C1 | N7 |
| 8 | C1 | N8 |
| 9 | C1 | N9 |
| 10 | C1 | N10 |
| 11 | C1 | N11 |
| 12 | C1 | N12 |
| 13 | C1 | N13 |
| 14 | C1 | N14 |
| 15 | C1 | N15 |
| 16 | C1 | N16 |
| 17 | C1 | N17 |
| 18 | C1 | N18 |
| 19 | C1 | N19 |
| 20 | C1 | N20 |
| 21 | C1 | N21 |
| 22 | C1 | N22 |
| 23 | C1 | N23 |
| 24 | C1 | N24 |
| 25 | C1 | N25 |
| 26 | C1 | N26 |
| 27 | C1 | N27 |
| 28 | C2 | N1 |
| 29 | C2 | N2 |
| 30 | C2 | N3 |
| 31 | C2 | N5 |
| 32 | C2 | N6 |
| 33 | C2 | N7 |
| 34 | C2 | N8 |
| 35 | C2 | N9 |
| 36 | C2 | N10 |
| 37 | C2 | N12 |
| 38 | C2 | N13 |
| 39 | C2 | N14 |
| 40 | C2 | N15 |
| 41 | C2 | N16 |
| 42 | C2 | N18 |
| 43 | C2 | N20 |
| 44 | C2 | N21 |
| 45 | C2 | N22 |
| 46 | C2 | N23 |
| 47 | C2 | N24 |
| 48 | C2 | N25 |
| 49 | C2 | N26 |
| 50 | C2 | N27 |
| 51 | C3 | N1 |
| 52 | C3 | N2 |
| 53 | C3 | N3 |
| 54 | C3 | N5 |
| 55 | C3 | N6 |
| 56 | C3 | N7 |
| 57 | C3 | N8 |
| 58 | C3 | N9 |
| 59 | C3 | N10 |
| 60 | C3 | N12 |
| 61 | C3 | N13 |
| 62 | C3 | N14 |
| 63 | C3 | N15 |
| 64 | C3 | N16 |
| 65 | C3 | N18 |
| 66 | C3 | N20 |
| 67 | C3 | N21 |
| 68 | C3 | N22 |
| 69 | C3 | N23 |
| 70 | C3 | N24 |
| 71 | C3 | N25 |
| 72 | C3 | N26 |
| 73 | C3 | N27 |
| 74 | C4 | N1 |
| 75 | C4 | N2 |
| 76 | C4 | N3 |
| 77 | C4 | N5 |
| 78 | C4 | N6 |
| 79 | C4 | N7 |
| 80 | C4 | N8 |
| 81 | C4 | N9 |
| 82 | C4 | N10 |
| 83 | C4 | N12 |
| 84 | C4 | N13 |
| 85 | C4 | N14 |
| 86 | C4 | N15 |
| 87 | C4 | N16 |
| 88 | C4 | N18 |
| 89 | C4 | N20 |
| 90 | C4 | N21 |
| 91 | C4 | N22 |
| 92 | C4 | N23 |
| 93 | C4 | N24 |
| 94 | C4 | N25 |
| 95 | C4 | N26 |
| 96 | C4 | N27 |
| 97 | C5 | N1 |
| 98 | C5 | N2 |
| 99 | C5 | N3 |
| 100 | C5 | N5 |
| 101 | C5 | N6 |
| 102 | C5 | N7 |
| 103 | C5 | N8 |
| 104 | C5 | N9 |
| 105 | C5 | N10 |
| 106 | C5 | N12 |
| 107 | C5 | N13 |
| 108 | C5 | N14 |
| 109 | C5 | N15 |
| 110 | C5 | N16 |
| 111 | C5 | N18 |
| 112 | C5 | N20 |
| 113 | C5 | N21 |
| 114 | C5 | N22 |
| 115 | C5 | N23 |
| 116 | C5 | N24 |
| 117 | C5 | N25 |
| 118 | C5 | N26 |
| 119 | C5 | N27 |
| 120 | C6 | N1 |
| 121 | C6 | N2 |
| 122 | C6 | N3 |
| 123 | C6 | N4 |
| 124 | C6 | N5 |
| 125 | C6 | N6 |
| 126 | C6 | N7 |
| 127 | C6 | N8 |
| 128 | C6 | N9 |
| 129 | C6 | N10 |
| 130 | C6 | N11 |
| 131 | C6 | N12 |
| 132 | C6 | N13 |
| 133 | C6 | N14 |
| 134 | C6 | N15 |
| 135 | C6 | N16 |
| 136 | C6 | N17 |
| 137 | C6 | N18 |
| 138 | C6 | N19 |
| 139 | C6 | N20 |
| 140 | C6 | N21 |
| 141 | C6 | N22 |
| 142 | C6 | N23 |
| 143 | C6 | N24 |
| 144 | C6 | N25 |
| 145 | C6 | N26 |
| 146 | C6 | N27 |
| 147 | C7 | N1 |
| 148 | C7 | N2 |
| 149 | C7 | N3 |
| 150 | C7 | N4 |
| 151 | C7 | N5 |
| 152 | C7 | N6 |
| 153 | C7 | N7 |
| 154 | C7 | N8 |
| 155 | C7 | N9 |
| 156 | C7 | N10 |
| 157 | C7 | N11 |
| 158 | C7 | N12 |
| 159 | C7 | N13 |
| 160 | C7 | N14 |
| 161 | C7 | N15 |
| 162 | C7 | N16 |
| 163 | C7 | N17 |
| 164 | C7 | N18 |
| 165 | C7 | N19 |
| 166 | C7 | N20 |
| 167 | C7 | N21 |
| 168 | C7 | N22 |
| 169 | C7 | N23 |
| 170 | C7 | N24 |
| 171 | C7 | N25 |
| 172 | C7 | N26 |
| 173 | C7 | N27 |
| 174 | C23 | N1 |
| 175 | C23 | N2 |
| 176 | C23 | N3 |
| 177 | C23 | N5 |
| 178 | C23 | N6 |
| 179 | C23 | N7 |
| 180 | C23 | N8 |
| 181 | C23 | N9 |
| 182 | C23 | N10 |
| 183 | C23 | N12 |
| 184 | C23 | N13 |
| 185 | C23 | N14 |
| 186 | C23 | N15 |
| 187 | C23 | N16 |
| 188 | C23 | N18 |
| 189 | C23 | N20 |
| 190 | C23 | N21 |
| 191 | C23 | N22 |
| 192 | C23 | N23 |
| 193 | C23 | N24 |
| 194 | C23 | N25 |
| 195 | C23 | N26 |
| 196 | C23 | N27 |
| 197 | C44 | N1 |
| 198 | C44 | N2 |
| 199 | C44 | N3 |
| 200 | C44 | N5 |
| 201 | C44 | N6 |
| 202 | C44 | N7 |
| 203 | C44 | N8 |
| 204 | C44 | N9 |
| 205 | C44 | N10 |
| 206 | C44 | N12 |
| 207 | C44 | N13 |
| 208 | C44 | N14 |
| 209 | C44 | N15 |
| 210 | C44 | N16 |
| 211 | C44 | N18 |
| 212 | C44 | N20 |
| 213 | C44 | N21 |
| 214 | C44 | N22 |
| 215 | C44 | N23 |
| 216 | C44 | N24 |
| 217 | C44 | N25 |
| 218 | C44 | N26 |
| 219 | C44 | N27 |
| 220 | C46 | N1 |
| 221 | C46 | N2 |
| 222 | C46 | N3 |
| 223 | C46 | N4 |
| 224 | C46 | N5 |
| 225 | C46 | N6 |
| 226 | C46 | N7 |
| 227 | C46 | N8 |
| 228 | C46 | N9 |
| 229 | C46 | N10 |
| 230 | C46 | N11 |
| 231 | C46 | N12 |
| 232 | C46 | N13 |
| 233 | C46 | N14 |
| 234 | C46 | N15 |
| 235 | C46 | N16 |
| 236 | C46 | N17 |
| 237 | C46 | N18 |
| 238 | C46 | N19 |
| 239 | C46 | N20 |
| 240 | C46 | N21 |
| 241 | C46 | N22 |
| 242 | C46 | N23 |
| 243 | C46 | N24 |
| 244 | C46 | N25 |
| 245 | C46 | N26 |
| 246 | C46 | N27 |
| 247 | C66 | N1 |
| 248 | C66 | N2 |
| 249 | C66 | N3 |
| 250 | C66 | N4 |
| 251 | C66 | N5 |
| 252 | C66 | N6 |
| 253 | C66 | N7 |
| 254 | C66 | N8 |
| 255 | C66 | N9 |
| 256 | C66 | N10 |
| 257 | C66 | N11 |
| 258 | C66 | N12 |
| 259 | C66 | N13 |
| 260 | C66 | N14 |
| 261 | C66 | N15 |
| 262 | C66 | N16 |
| 263 | C66 | N17 |
| 264 | C66 | N18 |
| 265 | C66 | N19 |
| 266 | C66 | N20 |
| 267 | C66 | N21 |
| 268 | C66 | N22 |
| 269 | C66 | N23 |
| 270 | C66 | N24 |
| 271 | C66 | N25 |
| 272 | C66 | N26 |
| 273 | C66 | N27 |
| 274 | C67 | N1 |
| 275 | C67 | N2 |
| 276 | C67 | N3 |
| 277 | C67 | N4 |
| 278 | C67 | N5 |
| 279 | C67 | N6 |
| 280 | C67 | N7 |
| 281 | C67 | N8 |
| 282 | C67 | N9 |
| 283 | C67 | N10 |
| 284 | C67 | N11 |
| 285 | C67 | N12 |
| 286 | C67 | N13 |
| 287 | C67 | N14 |
| 288 | C67 | N15 |
| 289 | C67 | N16 |
| 290 | C67 | N17 |
| 291 | C67 | N18 |
| 292 | C67 | N19 |
| 293 | C67 | N20 |
| 294 | C67 | N21 |
| 295 | C67 | N22 |
| 296 | C67 | N23 |
| 297 | C67 | N24 |
| 298 | C67 | N25 |
| 299 | C67 | N26 |
| 300 | C67 | N27 |
| 301 | a chromene | N1 |
| | COX-2 | |
| | inhibitor | |
| 302 | a chromene | N2 |
| | COX-2 | |
| | inhibitor | |
| 303 | a chromene | N3 |
| | COX-2 | |
| | inhibitor | |
| 304 | a chromene | N4 |
| | COX-2 | |
| | inhibitor | |
| 305 | a chromene | N5 |
| | COX-2 | |
| | inhibitor | |
| 306 | a chromene | N6 |
| | COX-2 | |
| | inhibitor | |
| 307 | a chromene | N7 |
| | COX-2 | |
| | inhibitor | |
| 308 | a chromene | N8 |
| | COX-2 | |
| | inhibitor | |
| 309 | a chromene | N9 |
| | COX-2 | |
| | inhibitor | |
| 310 | a chromene | N10 |
| | COX-2 | |
| | inhibitor | |
| 311 | a chromene | N11 |
| | COX-2 | |
| | inhibitor | |
| 312 | a chromene | N12 |
| | COX-2 | |
| | inhibitor | |
| 313 | a chromene | N13 |
| | COX-2 | |
| | inhibitor | |
| 314 | a chromene | N14 |
| | COX-2 | |
| | inhibitor | |
| 315 | a chromene | N15 |
| | COX-2 | |
| | inhibitor | |
| 316 | a chromene | N16 |
| | COX-2 | |
| | inhibitor | |
| 317 | a chromene | N17 |
| | COX-2 | |
| | inhibitor | |
| 318 | a chromene | N18 |
| | COX-2 | |
| | inhibitor | |
| 319 | a chromene | N19 |
| | COX-2 | |
| | inhibitor | |
| 320 | a chromene | N20 |
| | COX-2 | |
| | inhibitor | |
| 321 | a chromene | N21 |
| | COX-2 | |
| | inhibitor | |
| 322 | a chromene | N22 |
| | COX-2 | |
| | inhibitor | |
| 323 | a chromene | N23 |
| | COX-2 | |
| | inhibitor | |
| 324 | a chromene | N24 |
| | COX-2 | |
| | inhibitor | |
| 325 | a chromene | N25 |
| | COX-2 | |
| | inhibitor | |
| 326 | a chromene | N26 |
| | COX-2 | |
| | inhibitor | |
| 327 | a chromene | N27 |
| | COX-2 | |
| | inhibitor | |
| 328 | C68 | N1 |
| 329 | C68 | N2 |
| 330 | C68 | N3 |
| 331 | C68 | N5 |
| 332 | C68 | N6 |
| 333 | C68 | N7 |
| 334 | C68 | N8 |
| 335 | C68 | N9 |
| 336 | C68 | N10 |
| 337 | C68 | N11 |
| 338 | C68 | N12 |
| 339 | C68 | N13 |
| 340 | C68 | N14 |
| 341 | C68 | N15 |
| 342 | C68 | N16 |
| 343 | C68 | N17 |
| 344 | C68 | N18 |
| 345 | C68 | N20 |
| 346 | C68 | N21 |
| 347 | C68 | N22 |
| 348 | C68 | N23 |
| 349 | C68 | N24 |
| 350 | C68 | N25 |
| 351 | C68 | N26 |
| 352 | C68 | N27 |

Biological Assays

### Evaluation of COX-1 and COX-2 Activity In Vitro

The COX-2 inhibiting agents of this invention exhibit inhibition in vitro of COX-2. The COX-2 inhibition activity of the compounds illustrated in the examples above are determined by the following methods. The COX-2 inhibition activity of the other COX-2 inhibitors of the present invention may also be determined by the following methods.

Preparation of Recombinant COX Baculoviruses

Recombinant COX-1 and COX-2 are prepared as described by Gierse et al, [J. Biochem., 305, 479-84 (1995)]. A 2.0 kb fragment containing the coding region of either human or murine COX-1 or human or murine COX-2 is cloned into a BamHI site of the baculovirus transfer vector pVL1393 (Invitrogen) to generate the baculovirus transfer vectors for COX-1 and COX-2 in a manner similar to the method of D. R. O'Reilly et al (Baculovirus Expression Vectors: A Laboratory Manual (1992)). Recombinant baculoviruses are isolated by transfecting 4 μg of baculovirus transfer vector DNA into SF9 insect cells (2×108) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull. 1555 (1987). Recombinant viruses are purified by three rounds of plaque purification and high titer (107-108 pfu/mL) stocks of virus are prepared. For large scale production, SF9 insect cells are infected in 10 liter fermentors (0.5×106/mL) with the recombinant baculovirus stock such that the multiplicity of infection is 0.1. After 72 hours the cells are centrifuged and the cell pellet is homogenized in Tris/Sucrose (50 mM: 25%, pH 8.0) containing 1% 3-[(3-cholamidopropyl)-dimethylammonio]-1-propanesulfonate (CHAPS). The homogenate is centrifuged at 10,000×G for 30 minutes, and the resultant supernatant is stored at −80° C. before being assayed for COX activity.

Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 AM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

Fast Assay for COX-1 and COX-2 Activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (0.05 M Potassium phosphate, pH 7.5, 2 μM phenol, 1 μM heme, 300 μM epinephrine) with the addition of 20 μl of 100 μM arachidonic acid (10 μM). Compounds are pre-incubated with the enzyme for 10 minutes at 25° C. prior to the addition of arachidonic acid. Any reaction between the arachidonic acid and the enzyme is stopped after two minutes at 37° C./room temperature by transferring 40 μl of reaction mix into 160 μl ELISA buffer and 25 μM indomethacin. The PGE2 formed is measured by standard ELISA technology (Cayman Chemical).

### Biological Evaluation

A combination therapy of a COX-2 inhibiting agent and a NMDA antagonist for the treatment or prevention of neuropathic pain in a mammal can be evaluated as described in the following tests. The tests compare the anti-algesic affects of the combinations of the present invention with their liability to induce motor impairment in rats.

### Sciatic Nerve Ligation Assay

For sciatic nerve ligation, male Sprague Dawley rats (180-2209) are anesthetized with isofluorane, the left sciatic nerve is exposed and 4 chromic catgut (4.0) ligatures are tied loosely around the nerve (spaced 1-2 min apart) immediately proximal to the point of trifurcation. In sham-operated animals, the same dissection is performed but without ligation.

Responses to mechanical pressure are assessed 7 days after ligation using a modified Randall-Selitto algesiometer in which constant force of 40 mmHg is applied to the hind paw and the latency to struggle is recorded as the reaction time. Mechanical allodynia is defined as the difference in reaction time for sham and ligature rats. Reaction times for drug treated rats are expressed as a percentage of this response.

Compounds are administered 1 h before the test.

### Carrageenan-Induced Hyperalgesia Assay

Male Sprague Dawley rats (100-120 g) receive an intraplantar injection of carrageenan (4.5 mg) and mechanical thresholds are determined 3 h later using a modified Ugo Basile Algesiometer. Control rats receive saline (0.15 ml l.pl.).

Hyperalgesia is defined as the difference in vocalisation threshold for saline- and carrageenan-injected rats. Paw pressure scores for drug-treated rats are expressed as a percentage of this response.

Compounds are administered 2 h after carrageenan.

### Motor-Coordination Assay

Male Sprague Dawley rats (160-180 g) are first trained to remain for 120 s on the rotarod apparatus revolving at 12 r.p.m. on the morning before the test. Animals then receive drug treatments and 1 h later are placed on an accelerating rotarod (increasing from 4-40 r.p.m. during a 5 min period) and the time the rats are able to remain on the rotarod is recorded.

The contents of each of the references cited herein, including the contents of the references cited within these primary references, are herein incorporated by reference in their entirety.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the methods, combinations and compositions of the present invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims that follow and that such claims be interpreted as broadly as is reasonable.

What is claimed is:

1. A composition comprising a COX-2 selective inhibitor and a NMDA receptor antagonist, wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a compound having a diarylmethylidenefuran, a compound having a 2-phenylaminobenzene acetic acid, a compound having a chromene, and parecoxib or a pharmaceutically acceptable salt, prodrug or isomer thereof; and
wherein the NMDA receptor antagonist is selected from the group consisting of
(−)-6,7-dichloro-1,4-dihydro-5-[3-(methoxymethyl)-5-(3-pyridinyl)-4H-1,2,4-triazol-4-yl]-2,3-quinoxalinedione,
(2R,4S)-rel-5,7-dichloro-1,2,3,4-tetrahydro-4-[[(phenylamino)carbonyl]amino]-2-quinolinecarboxylic acid,
(2R,6S)-1,2,3,4,5,6-hexahydro-3-[(2S)-2-methoxypropyl]-6,11,11-trimethyl-2,6-methano-3-benzazocin-9-ol,
(3E)-2-amino-4-(phosphonomethyl)-3-heptenoic acid,
(3R,4S)-rel-3,4-dihydro-3-[4-hydroxy-4-(phenylmethyl)-1-piperidinyl]-2H-1-benzopyran-4,7-diol,
(3S,4aR,6S,8aR)-decahydro-6-(phosphonomethyl)-3-isoquinolinecarboxylic acid,
3,6,7-tetrahydro-2,3-dioxo-N-phenyl-1H,5H-pyrido[1,2,3-de]quinoxaline-5-acetamide,
(αR)-α-amino-5-chloro-1-(phosphonomethyl)-1H-benzimidazole-2-propanoic acid,
[2-(8,9-dioxo-2,6-diazabicyclo[5.2.0]non-1(7)-en-2-yl)ethyl]-phosphonic acid,
[5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride,
1,4-dihydro-6-methyl-5-[(methylamino)methyl]-7-nitro-2,3-quinoxalinedione,
1-[2-(4-hydroxyphenoxy)ethyl]-4-[(4-methylphenyl)methyl]-4-piperidinol, hydrochloride,
1-[4-(1H-imidazol-4-yl)-3-butynyl]-4-(phenylmethyl)-piperidine,
1-aminocyclopentane-carboxylic acid,
2-[(2,3-dihydro-1H-inden-2-yl)amino]-acetamide, monohydrochloride,
2-hydroxy-5-[[(pentafluorophenyl)methyl]amino]-benzoic acid,
2-methyl-6-(phenylethynyl)-pyridine,
3-(phosphonomethyl)-L-phenylalanine,
3-[(1E)-2-carboxy-2-phenylethenyl]-4,6-dichloro-1H-indole-2-carboxylic acid,
4,6-dichloro-3-[(E)-(2-oxo-1-phenyl-3-pyrrolidinylidene)methyl]-1H-indole-2-carboxylic acid,
6-chloro-2,3,4,9-tetrahydro-9-methyl-2,3-dioxo-1H-indeno[1,2-b]pyrazine-9-acetic acid,
7-chlorothiokynurenic acid,
8-chloro-2,3-dihydropyridazino[4,5-b]quinoline-1,4-dione 5-oxide salt with 2-hydroxy-N,N,N-trimethyl-ethanaminium,
aptiganel,
besonprodil,
budipine,
conantokin G,
delucemine,
dexanabinol,
felbamate,
fluorofelbamate,
gacyclidine,
glycine,
ipenoxazone,
kaitocephalin,
lanicemine,
licostinel,
midafotel,
milnacipran,
N′-[2-chloro-5-(methylthio)phenyl]-N-methyl-N-[3-(methylthio)phenyl]-guanidine,
N′-[2-chloro-5-(methylthio)phenyl]-N-methyl-N-[3-[(R)-methylsulfinyl]phenyl]-guanidine,
neramexane,
orphenadrine,
remacemide,
topiramate,
α-amino-2-(2-phosphonoethyl)-cyclohexanepropanoic acid, and
α-amino-4-(phosphonomethyl)-benzeneacetic acid
or a pharmaceutically acceptable salt thereof.

2. The composition of claim 1 wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, and N-[2-(cyclohexyloxy)-4-nitrophenyl]methanesulfonamide or a pharmaceutically acceptable salt, prodrug or isomer thereof.

3. The composition of claim 1 wherein the COX-2 selective inhibitor is a compound having the structure 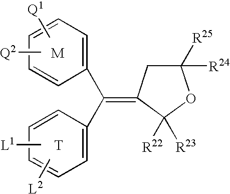
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²², R²³, R²⁴, and R²⁵ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²² and R²³ are O; or,
R²⁴ and R²⁵ are O; or,
R²², R²³, together with the carbon atom to which they are attached, form a saturated hydrocarbon ring having from 3 to 7 carbon atoms; or,
R²⁴, R²⁵, together with the carbon atom to which they are attached, form a saturated hydrocarbon ring having from 3 to 7 carbon atom;
or a pharmaceutically acceptable salt, an isomer or prodrug thereof.

4. The composition of claim 1 wherein the COX-2 selective inhibitor is a compound having the structure 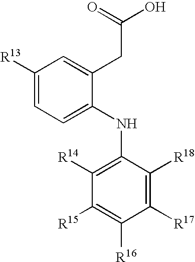
wherein:
R¹³ is methyl or ethyl;
R¹⁴ is chloro or fluoro;
R¹⁵ is hydrogen or fluoro;
R¹⁶ is hydrogen, fluoro, chloro, methyl, ethyl, methoxy, ethoxy or hydroxy;
R¹⁷ is hydrogen or fluoro; and
R¹⁸ is chloro, fluoro, trifluoromethyl or methyl, provided that R¹⁴, R¹⁷ and R¹⁸ are not all fluoro when R¹³ is ethyl and R¹⁶ is H;
or a pharmaceutically acceptable salt, an isomer or prodrug thereof.

5. The composition of claim 1 wherein the COX-2 selective inhibitor is a compound having the structure 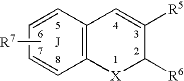
wherein:
X is selected from the group consisting of oxygen or sulfur or NR^{a};
R^{a} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring J forms a naphthyl radical,
or a pharmaceutically acceptable salt, an isomer or prodrug thereof.

6. The composition of claim 1 wherein the NMDA receptor antagonist is selected from the group consisting of
(−)-6,7-dichloro-1,4-dihydro-5-[3-(methoxymethyl)-5-(3-pyridinyl)-4H-1,2,4-triazol-4-yl]-2,3-quinoxalinedione;
1-aminocyclopentane-carboxylic acid;
4,6-dichloro-3-[(E)-(2-oxo-1-phenyl-3-pyrrolidinylidene)methyl]-1H-indole-2-carboxylic acid;
aptiganel;
besonprodil;
budipine;
conantokin G;
delucemine;
dexanabinol;
felbamate;
gacyclidine;
glycine;
ipenoxazone;
licostinel;
midafotel;
milnacipran;
N′-[2-chloro-5-(methylthio)phenyl]-N-methyl-N-[3-(methylthio)phenyl]-guanidine;
neramexane;
orphenadrine;
remacemide; and
topiramate
or a pharmaceutically acceptable salt thereof.

7. A method for the treatment or prevention of neuropathic pain in a subject, the method comprising administering to the subject a COX-2 selective inhibitor and a NMDA receptor antagonist,
wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, N-[2-(cyclohexyloxy)-4-nitrophenyl]methanesulfonamide, a compound having a diarylmethylidenefuran, a compound having a 2-phenylaminobenzene acetic acid, a compound having a chromene, and parecoxib or a pharmaceutically acceptable salt, prodrug or isomer thereof; and
wherein the NMDA receptor antagonist is selected from the group consisting of
(−)-6,7-dichloro-1,4-dihydro-5-[3-(methoxymethyl)-5-(3-pyridinyl)-4H-1,2,4-triazol-4-yl]-2,3-quinoxalinedione,
(2R,4S)-rel-5,7-dichloro-1,2,3,4-tetrahydro-4-[[(phenylamino)carbonyl]amino]-2-quinolinecarboxylic acid,
(2R,6S)-1,2,3,4,5,6-hexahydro-3-[(2S)-2-methoxypropyl]-6,11,11-trimethyl-2,6-methano-3-benzazocin-9-ol,
(3E)-2-amino-4-(phosphonomethyl)-3-heptenoic acid,
(3R,4S)-rel-3,4-dihydro-3-[4-hydroxy-4-(phenylmethyl)-1-piperidinyl]-2H-1-benzopyran-4,7-diol,
(3S,4aR,6S,8aR)-decahydro-6-(phosphonomethyl)-3-isoquinolinecarboxylic acid,
3,6,7-tetrahydro-2,3-dioxo-N-phenyl-1H,5H-pyrido[1,2,3-de]quinoxaline-5-acetamide,
(αR)-α-amino-5-chloro-1-(phosphonomethyl)-1H-benzimidazole-2-propanoic acid,
[2-(8,9-dioxo-2,6-diazabicyclo[5.2.0]non-1(7)-en-2-yl)ethyl]-phosphonic acid,
[5-(aminomethyl)-2-[[[(5S)-9-chloro-2,3,6,7-tetrahydro-2,3-dioxo-1H,5H-pyrido[1,2,3-de]quinoxalin-5-yl]acetyl]amino]phenoxy]-acetic acid, monohydrochloride,
1,4-dihydro-6-methyl-5-[(methylamino)methyl]-7-nitro-2,3-quinoxalinedione,
1-[2-(4-hydroxyphenoxy)ethyl]-4-[(4-methylphenyl)methyl]-4-piperidinol, hydrochloride,
1-[4-(1H-imidazol-4-yl)-3-butynyl]-4-(phenylmethyl)-piperidine,
1-aminocyclopentane-carboxylic acid,
2-[(2,3-dihydro-1H-inden-2-yl)amino]-acetamide, monohydrochloride,
2-hydroxy-5-[[(pentafluorophenyl)methyl]amino]-benzoic acid,
2-methyl-6-(phenylethynyl)-pyridine,
3-(phosphonomethyl)-L-phenylalanine,
3-[(1E)-2-carboxy-2-phenylethenyl]-4,6-dichloro-1H-indole-2-carboxylic acid,
4,6-dichloro-3-[(E)-(2-oxo-1-phenyl-3-pyrrolidinylidene)methyl]-1H-indole-2-carboxylic acid,
6-chloro-2,3,4,9-tetrahydro-9-methyl-2,3-dioxo-1H-indeno[1,2-b]pyrazine-9-acetic acid,
7-chlorothiokynurenic acid,
8-chloro-2,3-dihydropyridazino[4,5-b]quinoline-1,4-dione 5-oxide salt with 2-hydroxy-N,N,N-trimethyl-ethanaminium,
aptiganel,
besonprodil,
budipine,
conantokin G,
delucemine,
dexanabinol,
felbamate,
fluorofelbamate,
gacyclidine,
glycine,
ipenoxazone,
kaitocephalin,
lanicemine,
licostinel,
midafotel,
milnacipran,
N′-[2-chloro-5-(methylthio)phenyl]-N-methyl-N-[3-(methylthio)phenyl]-guanidine,
N′-[2-chloro-5-(methylthio)phenyl]-N-methyl-N-[3-[(R)-methylsulfinyl]phenyl]-guanidine,
neramexane,
orphenadrine,
remacemide,
topiramate,
α-amino-2-(2-phosphonoethyl)-cyclohexanepropanoic acid, and
α-amino-4-(phosphonomethyl)-benzeneacetic acid
or a pharmaceutically acceptable salt thereof.

8. The method of claim 7 wherein the COX-2 selective inhibitor is selected from the group consisting of celecoxib, deracoxib, valdecoxib, rofecoxib, etoricoxib, meloxicam, 4-(4-cyclohexyl-2-methyloxazol-5-yl)-2-fluorobenzenesulfonamide, 2-(3,5-difluorophenyl)-3-[4-(methylsulfonyl)phenyl]-2-cyclopenten-1-one, 2-(3,4-difluorophenyl)-4-(3-hydroxy-3-methylbutoxy)-5-[4-(methylsulfonyl)phenyl]-3(2H)-pyridazinone, and N-[2-(cyclohexyloxy)-4-nitrophenyl]methanesulfonamide or a pharmaceutically acceptable salt, prodrug or isomer thereof.

9. The method of claim 7 wherein the COX-2 selective inhibitor is a compound having the structure 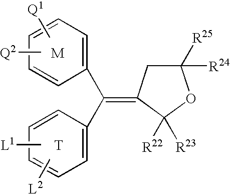
wherein:
T and M independently are phenyl, naphthyl, a radical derived from a heterocycle comprising 5 to 6 members and possessing from 1 to 4 heteroatoms, or a radical derived from a saturated hydrocarbon ring having from 3 to 7 carbon atoms;
Q¹, Q², L¹ or L² are independently hydrogen, halogen, lower alkyl having from 1 to 6 carbon atoms, trifluoromethyl, or lower methoxy having from 1 to 6 carbon atoms; and at least one of Q¹, Q², L¹ or L² is in the para position and is —S(O)ₙ—R, wherein n is 0, 1, or 2 and R is a lower alkyl radical having 1 to 6 carbon atoms or a lower haloalkyl radical having from 1 to 6 carbon atoms, or an —SO₂NH₂; or,
Q¹ and Q² are methylenedioxy; or
L¹ and L² are methylenedioxy; and
R²², R²³, R²⁴, and R²⁵ are independently hydrogen, halogen, lower alkyl radical having from 1 to 6 carbon atoms, lower haloalkyl radical having from 1 to 6 carbon atoms, or an aromatic radical selected from the group consisting of phenyl, naphthyl, thienyl, furyl and pyridyl; or,
R²² and R²³ are 0; or,
R²⁴ and R²⁵ are 0; or,
R²², R²³, together with the carbon atom to which they are attached, form a saturated hydrocarbon ring having from 3 to 7 carbon atoms; or,
R²⁴, R²⁵, together with the carbon atom to which they are attached, form a saturated hydrocarbon ring having from 3 to 7 carbon atom;
or a pharmaceutically acceptable salt, an isomer or prodrug thereof.

10. The method of claim 7 wherein the COX-2 selective inhibitor is a compound having the structure 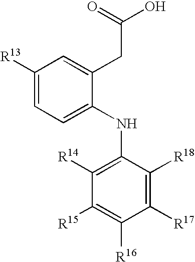
wherein:
R¹³ is methyl or ethyl;
R¹⁴ is chloro or fluoro;
R¹⁵ is hydrogen or fluoro;
R¹⁶ is hydrogen, fluoro, chloro, methyl, ethyl, methoxy, ethoxy or hydroxy;
R¹⁷ is hydrogen or fluoro; and
R¹⁸ is chloro, fluoro, trifluoromethyl or methyl,
provided that R, R R¹⁷ and R¹⁸ are not all fluoro when R¹³ is ethyl and R¹⁶ is H;
or a pharmaceutically acceptable salt, an isomer or prodrug thereof.

11. The method of claim 7 wherein the COX-2 selective inhibitor is a compound having the structure 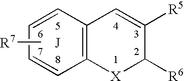
wherein:
X is selected from the group consisting of O or S or NR^{a};
R^{a} is alkyl;
R⁵ is selected from the group consisting of carboxyl, aminocarbonyl, alkylsulfonylaminocarbonyl and alkoxycarbonyl;
R⁶ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and
R⁷ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R⁷ together with ring J forms a naphthyl radical;
or a pharmaceutically acceptable salt, an isomer or prodrug thereof.

12. The method of claim 7 wherein the NMDA receptor antagonist is selected from the group consisting of
(−)-6,7-dichloro-1,4-dihydro-5-[3-(methoxymethyl)-5-(3-pyridinyl)-4H-1,2,4-triazol-4-yl]-2,3-quinoxalinedione;
1-aminocyclopentane-carboxylic acid;
4,6-dichloro-3-[(E)-(2-oxo-1-phenyl-3-pyrrolidinylidene)methyl]-1H-indole-2-carboxylic acid;
aptiganel;
besonprodil;
budipine;
conantokin G;
delucemine;
dexanabinol;
felbamate;
gacyclidine;
glycine;
ipenoxazone;
licostinel;
midafotel;
milnacipran;
N′-[2-chloro-5-(methylthio)phenyl]-N-methyl-N-[3-(methylthio)phenyl]-guanidine;
neramexane;
orphenadrine;
remacemide; and
topiramate
or a pharmaceutically acceptable salt thereof.

13. A composition comprising a COX-2 selective inhibitor and a NMDA receptor antagonist, wherein the COX-2 selective inhibitor is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a compound having a 2-phenylaminobenzene acetic acid, a compound having a chromene, and parecoxib or a pharmaceutically acceptable salt, isomer or prodrug thereof; and
wherein the NMDA receptor antagonist is selected from the group consisting of amantidine, dextromethorphan, ketamine, memantine, and traxoprodil or a pharmaceutically acceptable salt thereof.

14. A method for the treatment or prevention of neuropathic pain in a subject, the method comprising administering to the subject a COX-2 selective inhibitor and a NMDA receptor antagonist, wherein the COX-2 selective inhibitor is selected from the group consisting of valdecoxib, meloxicam, 4-(4-cyclohexyl-2-methyloxazol-5-yl)-2-fluorobenzenesulfonamide, 2-(3,4-difluorophenyl)-4-(3-hydroxy-3-methylbutoxy)-5-[4-(methylsulfonyl)phenyl]-3(2H)-pyridazinone, a compound having a 2-phenylaminobenzene acetic acid, a compound having a chromene, and parecoxib or a pharmaceutically acceptable salt, isomer or prodrug thereof; and
wherein the NMDA receptor antagonist is selected from the group consisting of amantidine, dextromethorphan, ketamine, memantine, and traxoprodil or a pharmaceutically acceptable salt thereof.

15. A composition comprising a COX-2 selective inhibitor and a NMDA receptor antagonist, wherein the COX-2 selective inhibitor is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide or a pharmaceutically acceptable salt, isomer or prodrug thereof; and
wherein the NMDA receptor antagonist is selected from the group consisting of amantidine, dextromethorphan, ketamine, and memantine or a pharmaceutically acceptable salt thereof.

16. A method for the treatment or prevention of neuropathic pain in a subject, the method comprising administering to the subject a COX-2 selective inhibitor and a NMDA receptor antagonist, wherein the COX-2 selective inhibitor is selected from the group consisting of deracoxib, etoricoxib, 2-(3,5-difluorophenyl)-3-(4-(methylsulfonyl)phenyl)-2-cyclopenten-1-one, and N-[2-(cyclohexyloxy)-4-nitrophenyl]methanesulfonamide or a pharmaceutically acceptable salt, isomer or prodrug thereof; and
wherein the NMDA receptor antagonist is selected from the group consisting of amantidine, dextromethorphan, ketamine, and memantine or a pharmaceutically acceptable salt thereof.

17. A composition comprising a COX-2 selective inhibitor and a NMDA receptor antagonist, wherein the COX-2 selective inhibitor is a compound having a diarylmethylidenefuran or a pharmaceutically acceptable salt, prodrug or isomer thereof; and
wherein the NMDA receptor antagonist is selected from the group consisting of amantidine, memantine, and traxoprodil or a pharmaceutically acceptable salt thereof.

18. A method for the treatment or prevention of neuropathic pain in a subject, the method comprising administering to the subject a COX-2 selective inhibitor and a NMDA receptor antagonist, wherein the COX-2 selective inhibitor is a compound having a diarylmethylidenefuran or a pharmaceutically acceptable salt, prodrug or isomer thereof; and
wherein the NMDA receptor antagonist is selected from the group consisting of amantidine, memantine, and traxoprodil or a pharmaceutically acceptable salt thereof.
